(19) United States
(12) Patent Application Publication
(10) Pub. No.: US 2007/0239199 A1
(43) Pub. Date: Oct. 11, 2007

(54) Inferior vena cava filter

(76) Inventor: Swaminathan Jayaraman, Fremont, CA (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/394,661

(22) Filed: Mar. 31, 2006

(51) Int. Cl. A61M 29/00

(52) U.S. Cl. 606/200

(57) ABSTRACT

The present invention relates to a vascular filter including a coiled wire formed of a shape memory material for implantation into a vessel. The vascular filter captures particulates within the blood flow in the vessel, without substantially interfering with the normal blood flow. Prior to implantation, the coiled wire is generally elongated and thereafter it reverts to a predetermined shape that is suitable for filtering the blood flow. The predetermined shape of the vascular filter includes a plurality of loops coaxially disposed about a longitudinal axis and has a conical portion and a cylindrical portion.

### CROSS-REFERENCE TO RELATED APPLICATIONS

This application is a Continuation in Part of U.S. patent application Ser. No. 11/084,946 filed Mar. 31, 2005, which is a Continuation in Part of U.S. patent application Ser. No. 10/939,660 filed Sep. 13, 2004, which in turn is a Divisional of U.S. patent application Ser. No. 09/739,830, filed Dec. 20, 2000 (now U.S. Pat. No. 6,790,218) which claims the benefit under 35 U.S.C. §§ 119(e) of Provisional Application No. 60/171,593 filed Dec. 23, 1999. The contents of each of these applications are incorporated by reference herein.

### FIELD OF THE INVENTION

The present invention relates generally to an implantable blood filter. In particular, the implantable blood filter of the present invention is formed of a wire which includes a shape memory alloy.

### BACKGROUND OF THE INVENTION

A pulmonary embolism is an obstruction of the pulmonary artery or one of its branches by a blood clot or other foreign substance. A pulmonary embolism can be caused by a blood clot which migrated into the pulmonary artery or one of its branches. Mechanical interruption of the inferior vena cava presents an effective method of preventing of pulmonary embolisms.

Vena cava filters are devices which are implanted in the inferior vena cava, providing a mechanical barrier. The filters are used to filter peripheral venous blood clots, which if remaining in the blood stream can migrate in the pulmonary artery or one of its branches and cause harm.

Conventional implantable blood filters employing a variety of geometries are known. Many are generally basket shaped, in order to provide adequate clot-trapping area while permitting sufficient blood flow. Also known are filters formed of various loops of wire, including some designed to partially deform the vessel wall in which they are implanted.

Along with their many functional shapes, conventional filters may include other features. For example, peripheral arms may be provided to perform a centering function so that a filter is accurately axially aligned with the vessel in which it is implanted. In order to prevent migration under the pressure induced by normal circulation, many filters have anchoring features. Such anchoring features may include hook, ridges, etc.

Many presently used vena cava filters are permanently implanted in the inferior vena cava and remain there for the duration of the patient's life or are removably implanted, but still which remain in position for long durations. As such, the filters can incur tissue ingrowth from the surrounding tissue, resulting in a decreased blood flow and in blood clots. While some permanent filters are designed to be percutaneously “retrievable”, they often become embedded as their anchoring features become endothelialized by the vessel wall and retrieval must be done surgically.

### SUMMARY OF THE INVENTION

The present invention relates to a vascular filter. The vascular filter includes a coil formed of a shape memory alloy, the member having a free bottom end and a free top end, a first predetermined unexpanded shape, and a second predetermined expanded shape. The unexpanded shape is substantially linear and the expanded shape is includes a cylindrical and a conical portion, each having a plurality of loops coaxially disposed about a longitudinal axis and where the conical loops progressively decreasing in diameter from one end of the device to the other. An exterior surface of the cylindrical portion includes barbs for stabilizing and securing the filter in a vessel.

In one embodiment, the loops of the conical coil having a constant pitch. Alternatively, the loops can form a substantially conical coil having a variable pitch.

The device may be formed of a shape memory nickel-titanium alloy, such as nitinol, and the member may be substantially arcuate in cross-section. The shape memory alloy may display a one-way shape memory effect, or a two-way shape memory effect.

In yet another embodiment, the shape memory alloy displays a superelastic effect at body temperature. Preferably, the shape memory alloy has an austenite finish temperature below body temperature, thereby permitting the device to have superelastic properties at body temperature.

The filter may include a plurality of layers. At least one layer may be formed of a passive memory material, and in another embodiment at least two layers may be formed of active memory materials.

In another embodiment, at least one of the layers is a wire formed of a shape memory material, and at least one of the layers is a braid formed of a shape memory material. Preferably, the plurality of layers includes at least two layers braided together or one layer surrounded by a braid.

The present invention also relates to a method of delivering a filter into a vessel. The method includes the steps of: providing a filter having a proximal portion, a transition portion, and a distal portion, and further having an initial length; placing the coil in a removable sheath for delivery to the vessel; withdrawing a portion of the movable sheath from the allowing the distal portion of the filter to emerge from the sheath; and allowing the filter to expand.

### BRIEF DESCRIPTION OF THE DRAWINGS

Preferred features of the present invention are disclosed in the accompanying drawings, wherein similar reference characters denote similar elements throughout the several views, and wherein:

FIG. 1 is a perspective view of one embodiment of a conically coiled member according to the present invention;

FIG. 2 is a side view of the conically coiled member of FIG. 1;

FIG. 3 is another side view of the conically coiled member of FIG. 2 rotated clockwise 180°;

FIG. 4 is another side view of the conically coiled member of FIG. 2 rotated counterclockwise 90°;

FIG. 5 is another side view of the conically coiled member of FIG. 2 rotated clockwise 90°;

FIG. 6 is a top view of the conically coiled member of FIG. 2;

FIG. 7 is a bottom view of the conically coiled member of FIG. 2;

FIG. 8 is a perspective view of an alternate embodiment of a coiled member according to the present invention and having a configuration combining a conical portion, a cylindrical portion, and a generally linear portion;

FIG. 9 is a side view of the coiled member of FIG. 8;

FIG. 10 is another side view of the coiled member of FIG. 9 rotated counterclockwise 180°;

FIG. 11 is another side view of the coiled member of FIG. 9 rotated counterclockwise 90°;

FIG. 12 is another side view of the coiled member of FIG. 9 rotated clockwise 90°;

FIG. 13 is a bottom view of the coiled member of FIG. 9;

FIG. 14 is a top view of the coiled member of FIG. 9;

FIG. 15 is a collection of top views of various embodiments of coiled members according to the present invention, including (a)-(b) coils with loops that are not all coaxial about a central axis, (c) a coil with a lower, crooked anchor or clip section, (d)-(e) coils having lower anchors or clips with complex curvature, (f)-(k) coils having lower anchors or clips in fan or star-like configurations;

FIG. 16 is a perspective view of an alternate embodiment of a coiled member according to the present invention and having 1.5 loops;

FIG. 17 is a top view of another alternate embodiment of a coiled member according to the present invention;

FIG. 18 is a perspective view of the coiled member of FIG. 17;

FIG. 19 is a side view of another alternate embodiment of a coiled member according to the present invention;

FIG. 20 is another embodiment of a coiled member according to the present invention, rotated in various orientations;

FIG. 21 is another alternate embodiment of a coiled member according to the present invention, rotated in various orientations;

FIG. 22 is another embodiment of a coiled member according to the present invention, shown in (a) side view, (b) top view, (c) side view, and (d) perspective view;

FIG. 22A is another embodiment of a coiled member according to the present invention, shown in side view;

FIG. 23 is another embodiment of a coiled member according to the present invention, shown in (a) side view of the extended state, (b) side view of the final shape, and (c) perspective view of the final shape;

FIG. 24 is another embodiment according to the present invention, showing a sheath-based coil delivery system with partial side views of (a) the sheath and coil extended through an anatomical defect in tissue, (b) the sheath partially withdrawn and a portion of the coil exposed, and (c) the sheath completely withdrawn with the coil fully exposed;

FIG. 25(a) is a side view of a member formed of two layers;

FIG. 25(b) is a cross-sectional view of a braid portion disposed around a central core;

FIG. 26 is a side view of a composite coil configuration of the present invention;

FIG. 27 is a side view of a composite coil configuration of the present invention including an intertwined coil;

FIG. 28 depicts a coil member having lower anchors or clips in fan or star-like configurations;

FIG. 29 is a side view of a central hub member that can be used to couple different sections of a composite coil;

FIGS. 30A-B depict substantially linear members with a central hub member;

FIG. 31 depicts a composite coil using the central hub member of FIG. 29;

FIG. 32 depicts a central hub member with a neck portion;

FIG. 33 depicts a central hub member coupled to a secondary hub member;

FIG. 34 depicts of a central hub member of FIG. 33 including a coil member attached to the secondary hub member;

FIG. 35 depicts a coil having woven fibers there around;

FIG. 36 depicts a side view of a filter of the present invention;

FIG. 37 depicts a partial view of the cylindrical portion of the filter including barbs;

FIG. 38 depicts the filter of FIG. 36 positioned in a vessel;

FIG. 39 depicts a sectional view of the filter of FIG. 36 including an outer coating;

FIG. 40 depicts a sectional view of the filter of FIG. 36 include multiple layers;

FIG. 41 depicts a cartridge used for inserting the filter of FIG. 36;

FIG. 42 depicts a first insertion orientation of the filter of FIG. 36;

FIG. 43 depicts a second insertion orientation of the filter of FIG. 36;

FIG. 44 depicts a partial view of a retractable catheter for inserting the filter of FIG. 36;

FIG. 45 depicts the retractable catheter of FIG. 44 in an open condition;

FIG. 46 depicts a side view of an alternative filter of the present invention;

FIG. 47 depicts the filter of FIG. 36 positioned in the aortic arch;

FIG. 48 depicts filters of FIG. 36 positioned in the brachiocephalic artery and the left common carotid artery of the aortic arch;

FIG. 49 depicts a wire coil of the present invention used to repair an anatomic junction;

FIG. 50 depicts an exterior view of a repaired anatomic junction;

FIG. 51 depicts an isometric view of another filter of the present invention;

FIG. 52 depicts a curved wire form of the filter of FIG. 51;

FIG. 53 depicts an isometric view of another filter of the present invention;

FIG. 54 depicts a partial sectional isometric view of the filter of FIG. 53;

FIG. 55 depicts an S-shaped wire form of the filter of FIG. 53;

FIG. 57 depicts a front view of the filter of FIG. 53;

FIG. 57 depicts a side view of the filter of FIG. 53;

FIG. 58 depicts an isometric view of another filter of the present invention;

FIG. 59 depicts a second isometric view of the filter of FIG. 58;

FIG. 60 depicts a front view of the filter of FIG. 58; and

FIG. 61 depicts a side view of the filter of FIG. 58

### DETAILED DESCRIPTION OF THE INVENTION

In the description which follows, any reference to either direction or orientation is intended primarily and solely for purposes of illustration and is not intended in any way as a limitation to the scope of the present invention. Also, the particular embodiments described herein, although being preferred, are not to be considered as limiting of the present invention.

In prior applications, the shape memory alloy members of the present invention have been described as vasoocclusive devices for filling or blocking anatomical defects, such as openings, in the vascular tree, e.g., holes in veins, arteries or the heart of a mammal. The coil portion of the device is placed or allowed to extend within the opening, where it is contacted by blood. Blood thrombosis upon contact with the coil thus fills in open areas to prevent further blood transport through the defect. However, the shape memory alloy members of the present invention can also be used as fillers.

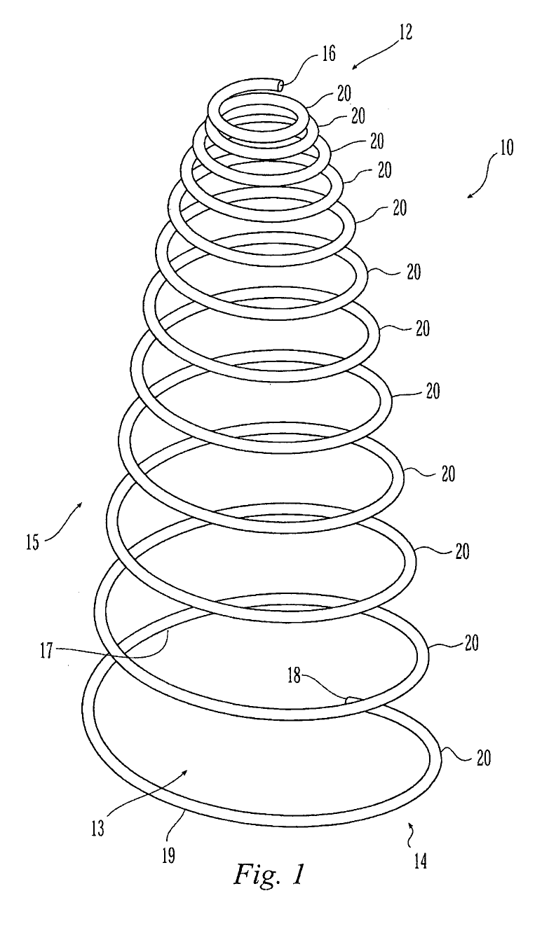

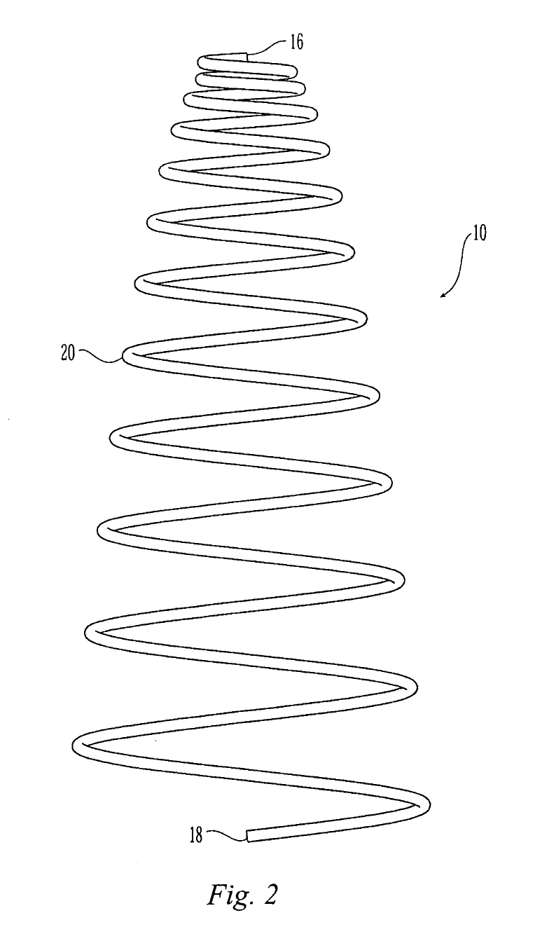

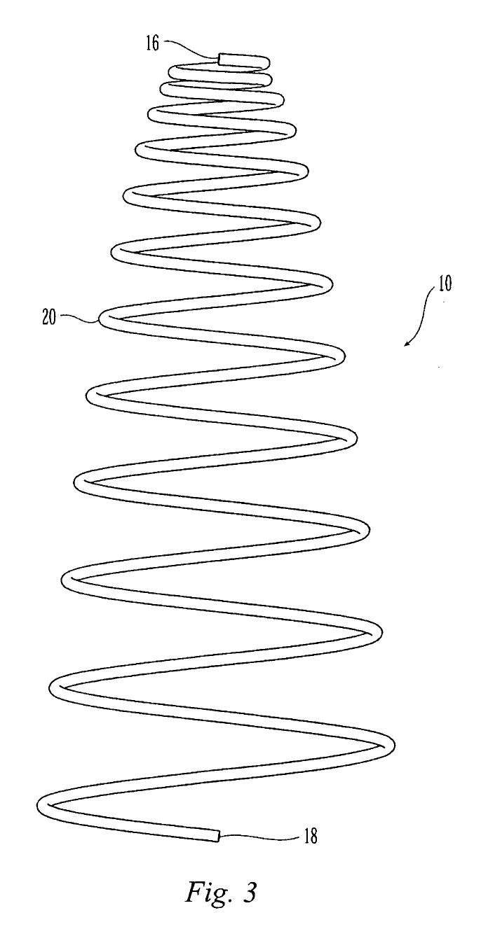

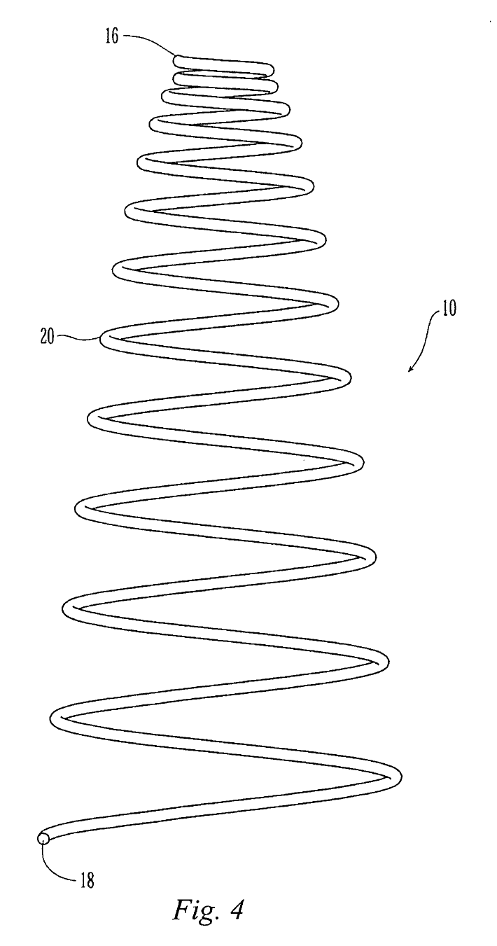

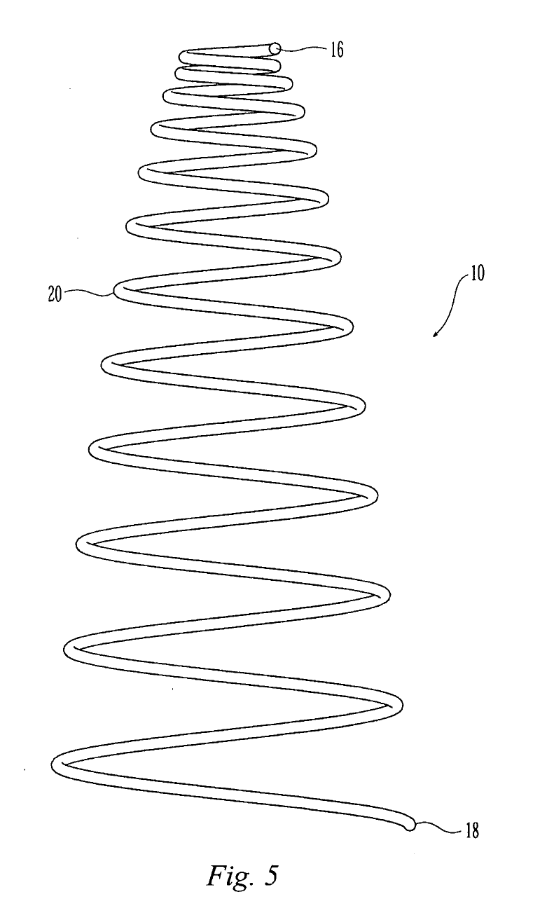

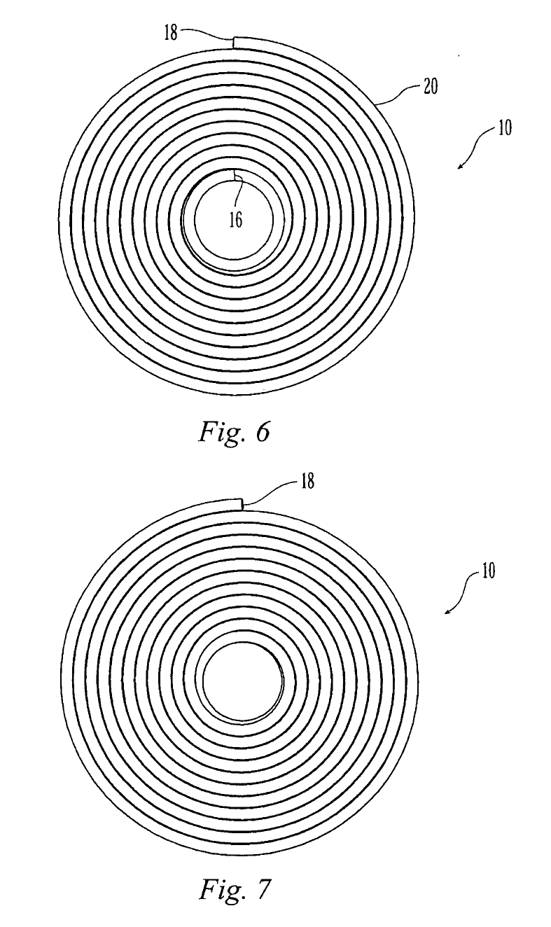

Referring to FIG. 1, there is shown a device or coil 10 that is formed in a conical spring configuration with a top end portion 12 and a bottom end portion 14. The coil 10 has a generally helical or spiral form. The top end 16 and bottom end 18 are joined by a series of loops 20. The loops 20 are coaxially disposed about a central longitudinal axis extending from the bottom end portion 14 to the top end portion 12. Coil 10 defines an inner area 13 and an outer area 15, the coil also having an inner surface 17 and outer surface 19 along each loop. In the embodiment illustrated in FIG. 1, the loops 20 decrease in diameter as they progress from the bottom end 18 to the top end 16. The coil in this embodiment is substantially conical, because it may not assume a perfectly conical configuration. Various side views of coil 10 are shown in FIGS. 2-5. For example, the coil 10 in FIG. 3 is rotated from the position shown in FIG. 2 clockwise 180° about the longitudinal axis extending from the bottom end portion 14 to the top end portion 12. FIG. 4 results from a counterclockwise rotation of 90°, while FIG. 5 results from a clockwise rotation of 90°. FIGS. 6 and 7 show the coil 10 from the top and bottom, respectively.

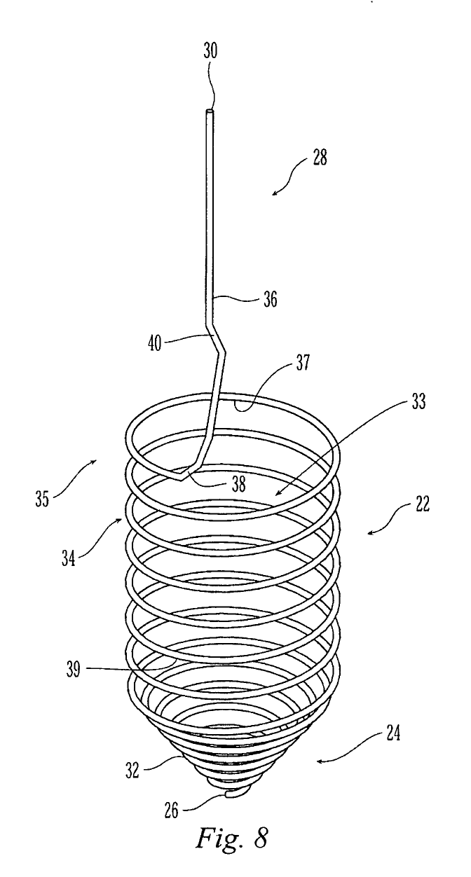

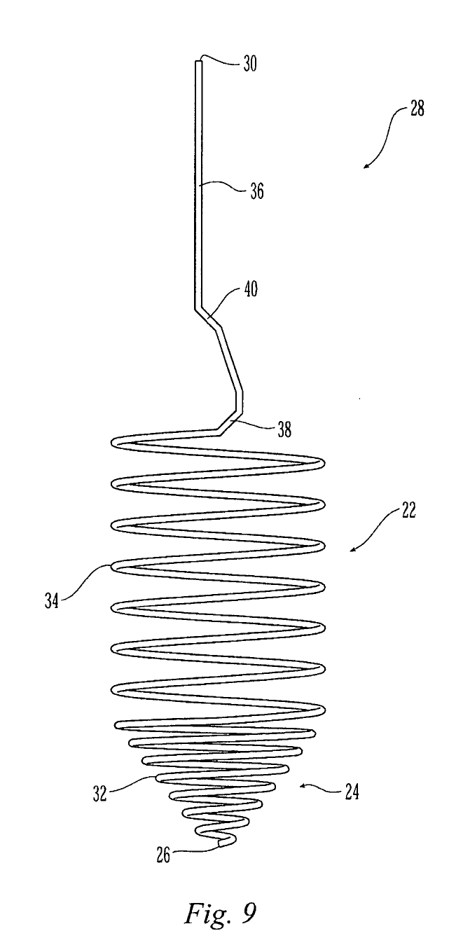

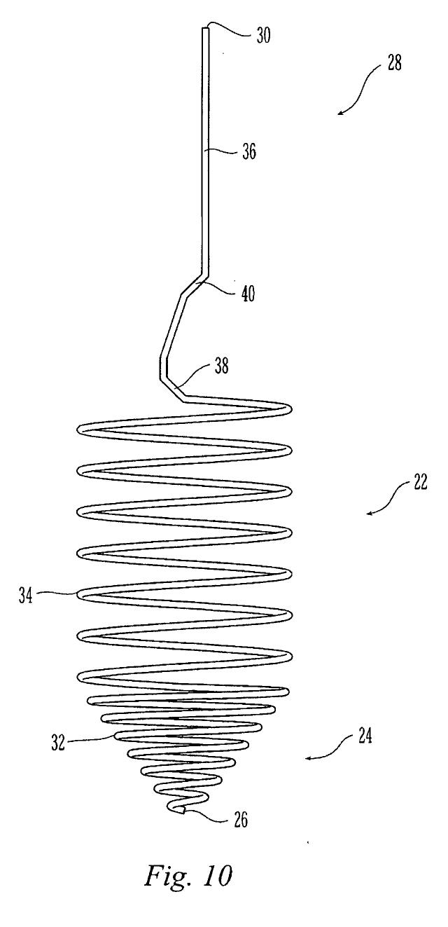

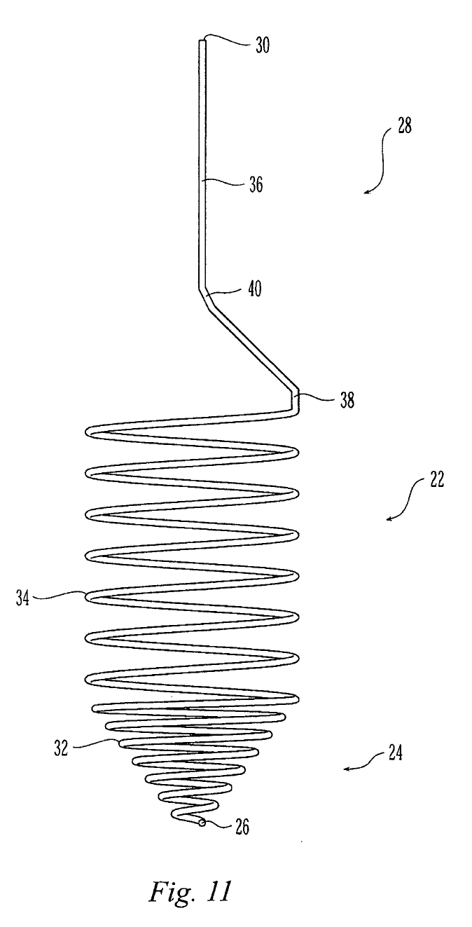

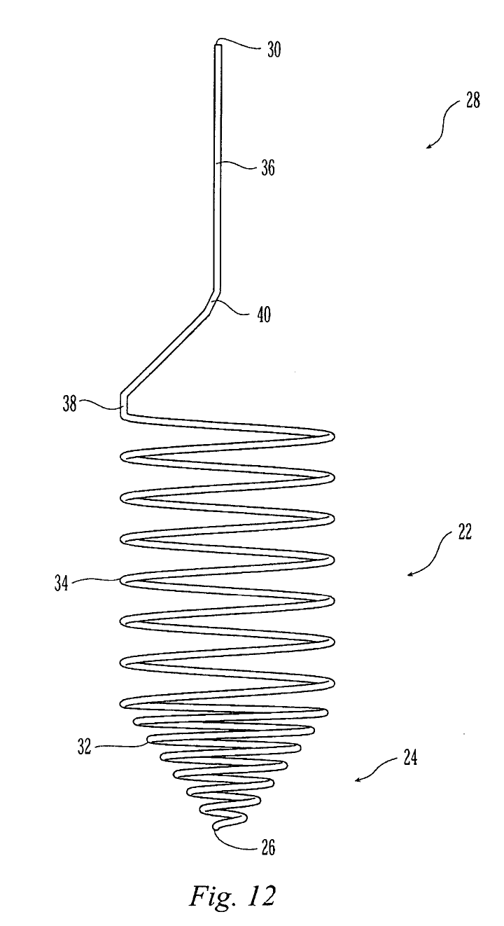

An alternative embodiment of the device 22 according to the present invention is shown in FIGS. 8-14. Device 22 includes an upper portion 24 having a top end 26 and a bottom portion 28 having a bottom end 30. Upper portion 24 has a substantially conical coiled section 32 followed by a substantially cylindrical section 34 and thereafter a generally linear section 36 that includes two crooked sections 38 and 40. The substantially conical and substantially cylindrical sections may not be precisely conical or cylindrical, respectively. As shown, the device 22 extends continuously from top end 26 to bottom end 30. Device 22 defines an inner area 33 and an outer area 35, the device also having an inner surface 37 and outer surface 39 along each loop. Various side views of device 22 are shown in FIGS. 9-13. For example, the device 22 in FIG. 10 is rotated from the position shown in FIG. 9 counterclockwise 180° about the longitudinal axis extending from the bottom portion 28 to the upper portion 24. FIG. 11 results from a counterclockwise rotation of 90°, while FIG. 12 results from a clockwise rotation of 90°. FIGS. 13 and 14 show the device 22 from the bottom and top, respectively.

In another alternate embodiment, not shown in the figures, the device 22 is substantially barrel shaped, or is provided with a substantially barrel shaped portion.

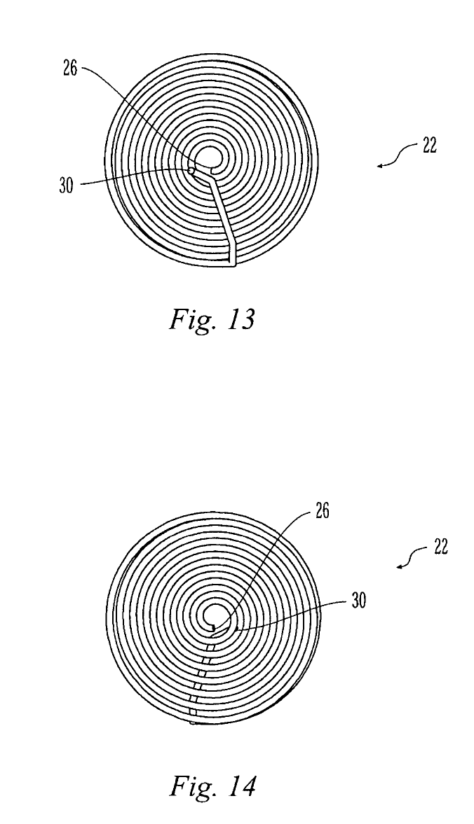

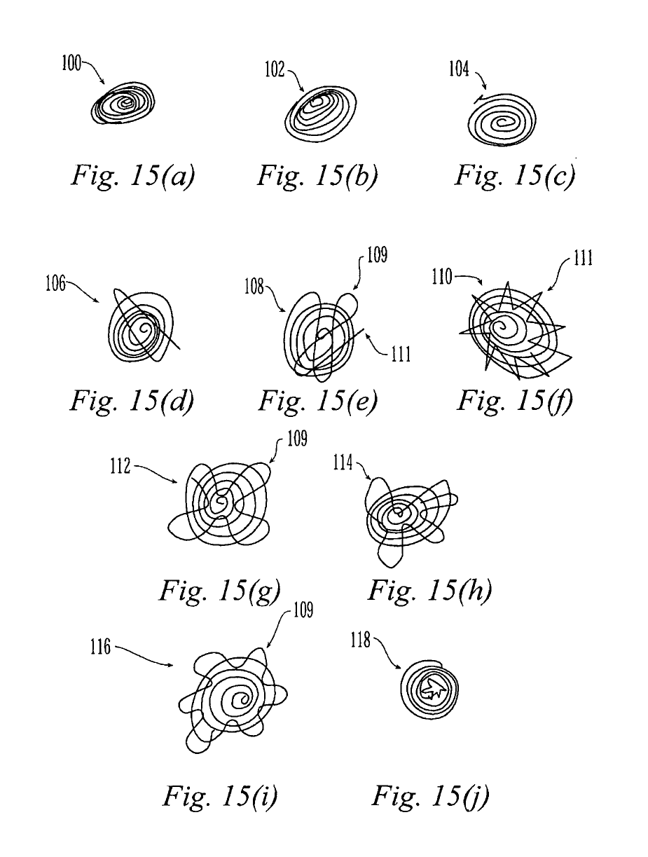

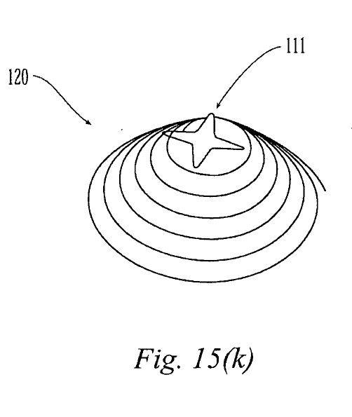

Various other configurations of coils according to the present invention are shown in FIG. 15. FIGS. 15(a)-(b) show coils 100 and 102, respectively, having loops that are not all coaxial about a central axis. FIG. 15(c) shows a coil 104 having a lower, crooked anchor section. FIGS. 15(d)-(e) show coils 106 and 108, respectively, having lower anchors with complex curvature. Also, FIGS. 15(f)-(k) show coils 110, 112, 114, 116, 118, and 120, respectively, having lower anchors or clips in fan or star-like configurations. Preferably, each clip has at least two prongs for contacting the tissue at a desired location. The prongs may be curved prongs 109 and/or sharp prongs 111. Advantageously, the use of prong configurations permits multiple anchor points to tissue, and thus also provides additional securing of the device.

The pitch of a coil, defined as the center-to-center distance between adjacent loops 20, may be constant or variable along the central longitudinal axis. The free length of the coil, defined as the overall length of the coil measured along the central longitudinal axis extending from the bottom end 18 to the top end 16, is chosen based on the geometry of the physiological parameters in question. Additionally, the coils may be right-handed or left-handed spirals. Furthermore, the decrease in diameter of the loops may be constant or variable.

In the preferred embodiment, the coil is not close-wound with adjacent loops 20 contacting each other. Instead, the loops 20 forming the ends 18 and 16 do not contact adjacent loops. Alternatively, the coil may be provided in close-wound form.

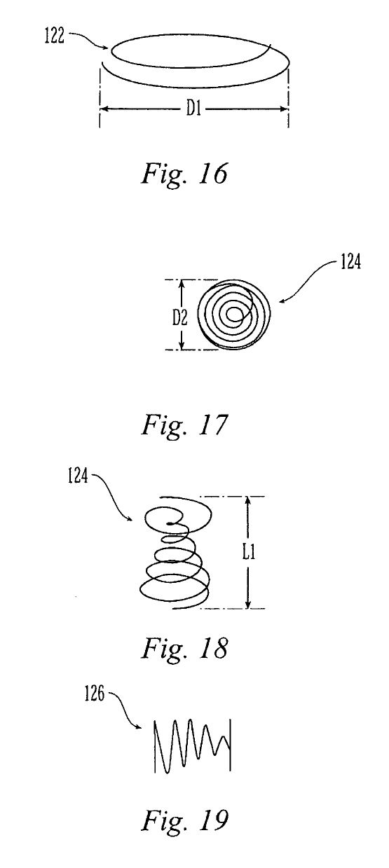

Another configuration of a coil according to the present invention is shown in FIG. 16. This coil 122 has only 1.5 loops. In a preferred embodiment, coil 122 has a maximum diameter of D₁ of 10 mm, and the total length of material used to form the coil is 44 mm. The radius of the full loop is different from the radius of the half loop. FIGS. 17-18 show yet another configuration of a coil according to the present invention. In a preferred embodiment, coil 124 has a maximum diameter of D₂ of 4.00 mm, and a maximum coiled length L₁ of 4.77 mm. In addition, the total length of material used to form coil 124 is 56 mm. Notably, the coil has a conical section with the smallest loop of the conical section also followed by a loop of larger diameter.

In another alternate embodiment shown in FIG. 19, a coil 126 has a generally conical profile, however the first and last loops each have a greater overall diameter than any of the intermediate loops.

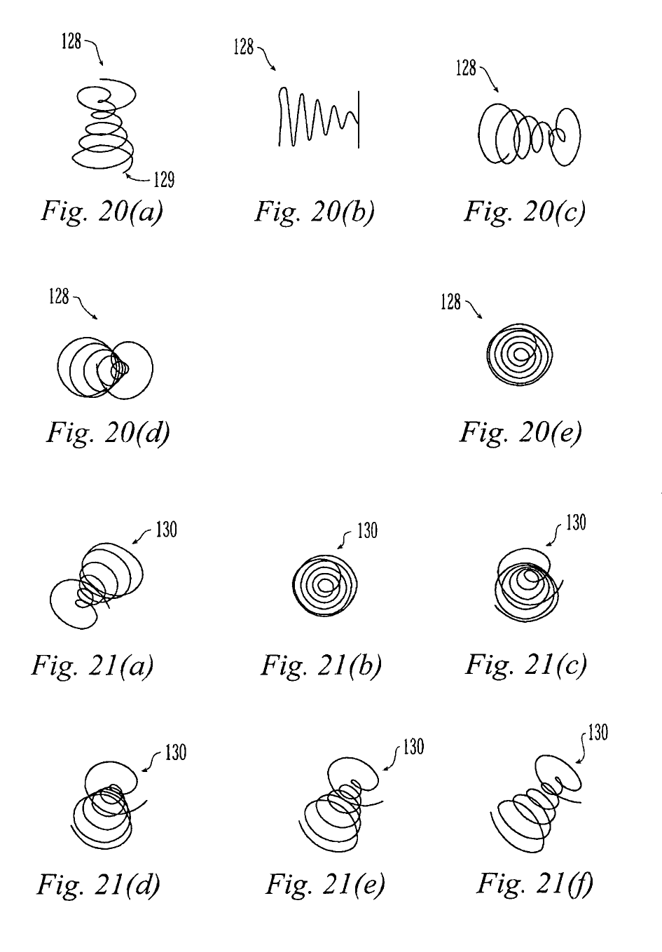

FIGS. 20 and 21 show two additional coils 128 and 130, respectively, according to the present development, each rotated in several orientations. Each coil includes an anchor portion that spirals away from the coil. An anchor portion 129 is clearly shown, for example, at the bottom of FIG. 20(a). However, either end of the coil may serve this function.

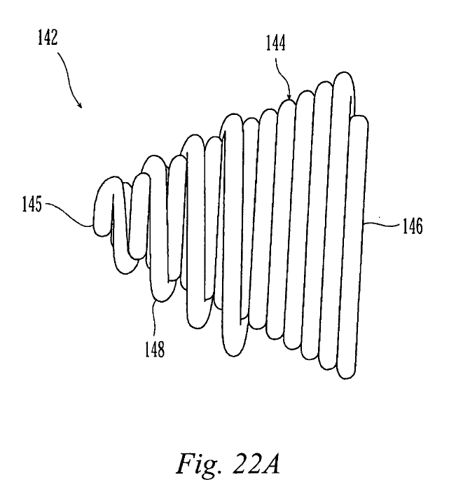

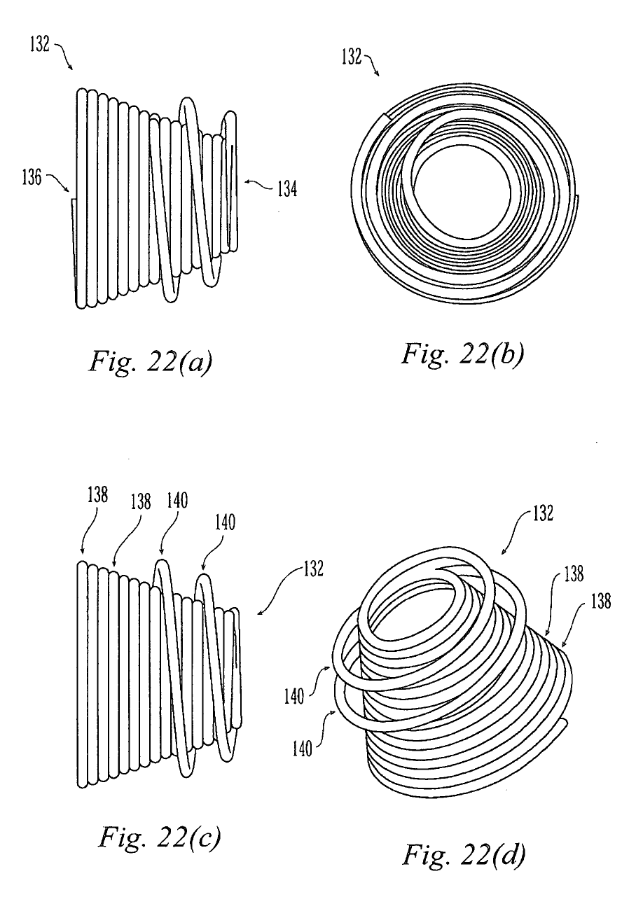

FIGS. 22(a)-(d) show another coil according to the present development. Coil 132 has a first end 134 and second end 136. Although coil 134 is generally conical in overall shape, several loops are formed toward first end 134 such that an inner set of loops 138 and an outer set of loops 140 are formed. The inner set of loops 138 at first end 134 have a smaller diameter than the inner set of loops 138 at second end 136.

In a variant of the coil shown in FIGS. 22(a)-(d), a coil 142 is shown in FIG. 22A with an inner set of loops 144 that form a cone from a first region 145 to a second region 146. An outer set of loops 148 also are provided, and extend from the narrow, first region 145. The inner set of loops 144 proximate first region 145 have a smaller diameter than the inner set of loops 144 at second region 146. In addition, in the embodiment as shown in FIG. 22A, the diameters of the outer set of loops 148 increase from the first region 145 toward the second region 146.

All embodiments of the coils may be adapted to include a clip on at least one of the coil ends. The clip enhances attachment of the coil to its surroundings. The clip may be a prong-like extension from the coil that has at least one generally straight section. Furthermore, the clip may be oriented transverse to the central longitudinal axis of the coil, or it may extend parallel to the axis. The choice of clip orientation may be partially determined by the anatomical features. Alternatively, the clip may be in the form of a lower anchor with an arcuate configuration, or a complex structure such as a star-like configuration.

The closure device is a coil made of a shape memory alloy. Such a material may be deformed at a temperature below a transition temperature region that defines a region of phase change, and upon heating above the transition temperature region assumes an original shape. The coil is preferably made of an alloy having shape-memory properties, including, but not limited to, the following alloys: Ni—Ti, Cu—Al—Ni, Cu—Zn, Cu—Zn—Al, Cu—Zn—Si, Cu—Sn, Cu—Zn—Sn, Ag—Cd, Au—Cd, Fe—Pt, Fe—Mn—Si, In—Ti, Ni—Al, and Mn—Cu. The coil is most preferably made of a nickel-titanium alloy. Such nickel-titanium alloys have gained acceptance in many medical applications, including stents used to reinforce vascular lumens.

NiTi alloys are particularly suitable for coils because of their shape memory and superelastic properties. These alloys have two temperature-dependent phases, the martensite or lower temperature phase, and the austenite or higher temperature phase. When the alloy is in the martensitic phase, it may be deformed due to its soft, ductile, and even rubber-like behavior. In the austenitic phase, the alloy is much stronger and rigid, although still reasonably ductile, and has a significantly higher Young's Modulus and yield strength. While the material transforms from one phase to the other, the transformation temperature range is dependent on whether the material is being heated or cooled. The martensite to austenite transformation occurs during heating, beginning at an austenite start temperature, Aₛ, and ending at an austenite finish temperature, A_{f}. Similarly, the austenite to martensite transformation occurs during cooling, beginning at a martensite start temperature, Mₛ, and ending at a martensite finish temperature, M_{f}. Notably, the transition temperatures differ depending on heating and cooling, behavior known as hysteresis.

Some alloys display a “one-way” shape memory effect; essentially, this is an ability of the material to have a stored, fixed configuration (sometimes referred to as a trained shape), that may be deformed to a different configuration at a temperature below the phase change region, and subsequently may be heated above the transition temperature region to reassume that original configuration. A select group of alloys also display a “two-way” shape memory effect, in which the material has a first, fixed configuration at low temperature, and a second, fixed configuration at temperatures above the phase change. Thus, in this case, the material may be trained to have two different shapes.

Superelasticity (sometimes referred to as pseudoelasticity) occurs over a temperature range generally beginning at A_{f}, and ending when the NiTi is further heated to a martensite deformation temperature, M_{d}, that marks the highest temperature at which a stress-induced martensite occurs. In some cases, superelasticity may be observed at temperatures extending below A_{f}. The superelasticity of the material in this temperature range permits the material to be deformed without plastic deformation, and thus permanent deformation is avoided.

In order to fix the shapes that the NiTi is to assume, a proper heat treatment must be applied. Depending on the application and the particular shape-memory or superelastic effect to be used, shapes may be fixed at each of the desired temperatures above or below the transitions.

The various transition temperatures and other materials properties of Ni—Ti may be tailored to the application in question. Due to the solubility of alloying elements in the nickel-titanium system, it is possible to deviate from a 50-50 ratio of nickel to titanium, by having either more nickel or titanium, or by adding alloying elements in relatively small quantities. Typical dopants include chromium, iron, and copper, although other elements may be selectively added to affect the properties. In addition, mechanical treatments, such as cold working, and heat treatments, such as annealing, may significantly change the various properties of the material.

Although the Ni-50% Ti shape memory alloy is generally referred to as nitinol, an abbreviation for Nickel Titanium Naval Ordnance Laboratory that recognizes the place of discovery, the term as used herein extends to nickel-titanium alloys that deviate from this ratio and that also may contain dopants.

The present invention also relates to a method of manufacturing coils and delivery of those coils. A substantially straight piece of nitinol wire may be introduced into specific regions of the body, and thereafter assumes a pre-set geometry. The delivery may take place through a sheath that serves a similar purpose to that of a catheter, or the temporarily straightened coil may be delivered through specific catheters. The wire remains straight until it is exposed to the inside of the body. Upon reaching the end of the delivery system, and warming to a temperature between 30° C. and 40° C., the normal body temperature, the wire may assume a predetermined shape. In a preferred embodiment, the wire assumes a shape as shown in FIG. 1, 8 or 15. The choice of shape depends on the length of the wire introduced, as well as the anatomy where it is introduced. Various shapes are contemplated, including circular forms, rectangular forms, offset coiled forms having loops that are not coaxially disposed about a longitudinal axis, and concentric coiled forms, although the shape is not limited to these embodiments. In a preferred embodiment, the shape is helical, conical, or spiral. The wire may assume any open ended shapes as a final configuration, with the exception of a straight line.

As noted, the dimensions and configuration of the coil depend on the anatomy. In a preferred embodiment, the maximum coil diameter is less than 1.5 cm. In another preferred embodiment, the sizes of the coil may be chosen as follows:

| | | | | | | |
|---|---|---|---|---|---|---|
| maximum coil diameter (mm) | 4 | 5 | 6 | 7 | 8 | 9 |
| diameter of the last loop (mm) | 3 | 3.5 | 4 | 5 | 6 | 6 |
| side profile width (mm) | 3 | 4 | 4 | 4 | 4 | 4 |

For each coil, the last loop may be provided with a back clip which is not conical in shape, and this clip attaches the coil to tissue. Preferably, during delivery of the coil, as it exits the delivery catheter it warms and assumes its predetermined loop-like configuration. If a clip is included with the coil, preferably the clip is released last from the catheter.

The device may be delivered via a 5 F (5 French) catheter that may be placed via a 6 F sheath. In its substantially straight configuration, the device should snugly fit in the catheter for slidable delivery.

The introduction device may also include a small metallic tube that initially completely houses the straightened device. The tube may be temporarily attached to the proximal end of the catheter, and the device may subsequently be inserted into the catheter with the help of a guidewire. The guidewire preferably is substantially straight, has a diameter similar to that of the wire used to form the coil, and additionally has a generally stiff end and a soft end. Once the device has been completely placed in the catheter, the tube is discarded, and the guidewire is used to place the device at the distal tip of the catheter and effect delivery of the device to the desired anatomical location.

Generally, if the device must be retrieved due to improper positioning, the retrieval must occur prior to delivery of the final loop section of the coil. Otherwise, a more complex coil removal procedure may be necessary. In order to facilitate coil delivery, radiopaque markers may be provided on the device, and preferably are provided on a top side at proximal and/or distal ends. In an alternate embodiment, markers may be provided continuously or in spaced, regular intervals along the length of the device. The use of such markers allows device delivery to be precisely monitored. Thus, if a device is not delivered properly to the chosen anatomical location, the device may be withdrawn into the sheath for re-release or may be completely withdrawn from the body.

In order for coil retrieval to occur, the coil is gripped at one end using a jaw or other retention mechanism as typically used with biopsy-related devices. Alternatively, other coil delivery and retrieval procedures involving pressure may be used, i.e. air pressure and suction. Prior to completion of coil delivery, if for example improper coil alignment has resulted or an improper coil shape or size has been chosen, the retention mechanism may be used to withdraw the coil into the sheath.

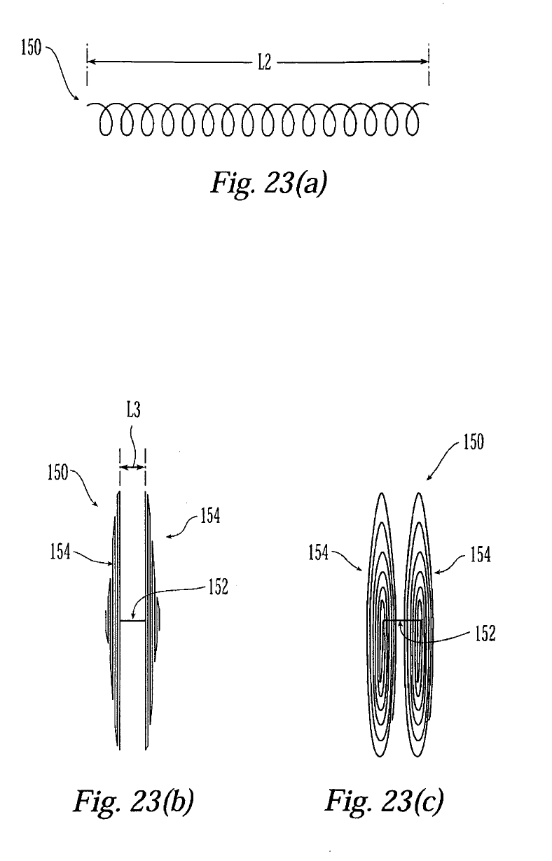

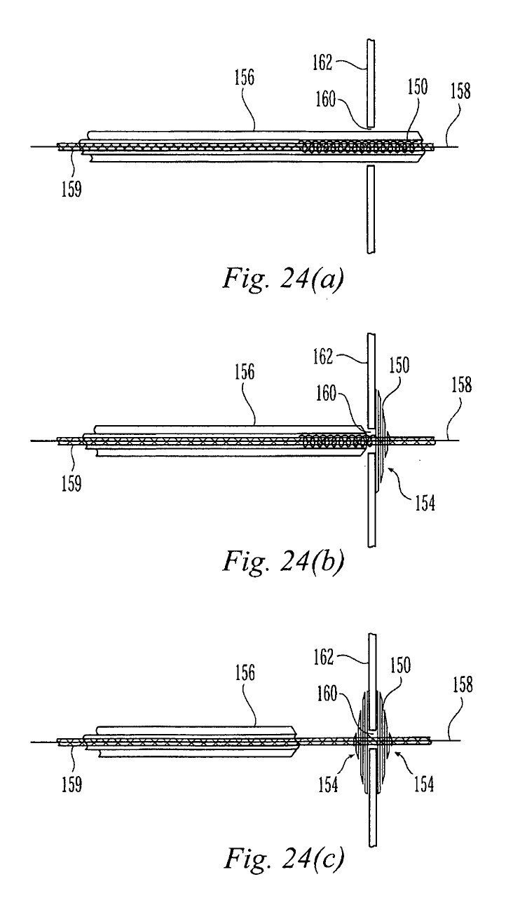

Alternatively, as shown in FIGS. 23-24, a coil 150 initially may be provided in an extended state such that its overall coiled length is L₂, and when delivered the coil assumes a final shape with an overall coiled length L₃. The final shape of coil 150 includes a transition section 152 between two spiral sections 154. Although the transition section 152 is generally straight in FIG. 23, transition section 152 may alternatively include loops forming a conical portion. Preferably, spiral sections 154 are formed such that the loops are generally coplanar. While coil movement may be constrained by a retention mechanism that, for example, grasps an end of a proximal portion of the coil, delivery of a coil such as coil 150 may be achieved using a movable sheath 156 and associated catheter.

A catheter may be used to deliver a coil 150 to an anatomical region. As shown in FIG. 24(a), a central shaft 158 is inserted through a hole 160 or other anatomical defect to be filled in tissue 162, which is depicted in partial side view. Such a hole 160, for example, may exist in a patient's heart in the septum. Central shaft 158 serves as a guidewire for the delivery of the coil. Preferably, central shaft 158 is surrounded by an inner sheath 159 formed of a braided metal wire having a layer of Teflon® (tetrafluoroethylene) on its inner surface for contacting central shaft 158 and a layer of Pebax® (polyether-block co-polyamide) on its outer surface for contacting coil 150. With central shaft 158 in place, an outer movable sheath 156 is extended through hole 160 using central shaft 158 as a guide. Preferably, outer movable sheath 156 is formed from polyethylene terephthalate (PET) or nylon. Coil 150 is disposed between inner sheath 159 and outer movable sheath 156. Coil 159 is wound about inner sheath 159, and restrained from expanding in the radial direction by outer movable sheath 156.

When outer movable sheath 156 is partially withdrawn, as shown in FIG. 24(b), a first, distal portion of coil 150 is exposed, warming to body temperature and thus assuming a preformed configuration. A first spiral section 154 forms on the far side of hole 160. Outer movable sheath 156 then may be further withdrawn, as shown in FIG. 24(c), exposing a transition portion of coil 150 and finally a proximal portion of coil 150 to the body, and thereby permitting coil 150 to assume the complete preformed configuration with a second spiral section 154 formed on the other, near side of hole 160. Coil 150 thus is held in place by the pressure applied by spiral sections 154 against tissue 162. A clip (not shown) also may be provided on one or both of spiral sections 154. A final coil release mechanism, such as a spring-release mechanism, may be used to separate coil 150 from the retention mechanism, and central shaft 158, inner sheath 159, and outer movable sheath 156 may be completely withdrawn from the body. A free end of coil 150 may be held by a biopsy forcep during the coil insertion procedure, to aid in the positioning and initial withdrawal of the sheath so that a spiral section 154 can be formed. In addition, the free ends of the coil may be capped or otherwise formed in the shape of beads. Such beads provide regions of increased thickness, and thus are detectable by x-ray equipment to aid in verification of coil positioning. The beads may also provide suitable structure for gripping by forceps. The sheath delivery method is particularly appropriate for the placement of coils having an overall length greater than twenty percent the length of the delivery catheter.

Several factors must be considered when choosing the size and shape of a coil to be used. The desired helical diameter of the coil, a measure of the final diameter of the coil after expansion to its circular shape and implantation, must be considered in light of the geometry. In addition, the length of the coil and the number of coil loops must be considered. Furthermore, coils may be designed with tightly packed windings, windings having only a short distance between each loop, or loosely packed windings having greater separation between neighboring loops. The length of the coil places an additional constraint on the number of loops that may be provided. Coils may be packaged and provided to the medical community based on any of the aforementioned factors, or a combination thereof.

In a preferred embodiment, the coils are provided based on the substantially straightened length of the wire and/or the number of coil loops. Alternatively, the coils may be provided for selection based on coil length and/or helical diameter. In a simple case, if all loops had the same diameter, for example, the circumference of a representative loop could be determined by multiplying the helical diameter by π. The number of loops could thus be determined by a supplier or medical practitioner by dividing the substantially straightened length by the circumference of the representative loop. In designs having variable loop diameters, the circumferences of the individual loops must be known in order to determine the number of loops for a given length of wire.

In general, the coil size can be chosen to have a helical diameter approximately 20% to 30% larger than the narrowest size of the vessel. Otherwise, distal migration may occur if the coil is too small, and coils that are too large may be unable to fully assume their intended final geometry. The coil caliber is determined by catheter size used to cannulate the vessel.

In general, the helical diameter of the coil can be 2 to 3 times the size of the narrowest point of the vessel. This is especially appropriate for duct sizes less than about 2.5 mm. However, multiple coils may be required. In particular, ducts greater than about 4 mm may require between 3 to 6 coils.

The wire used to form the coils preferably has an outer diameter of 0.018″, 0.025″, 0.035″, or 0.038″, and may be pre-loaded into a stainless steel or plastic tube for simple and direct insertion into the catheter or other delivery device. Several wires may be braided together in order to produce a wire with a desired outer diameter; for example, several wires each having outer diameters of approximately 0.010″ may be used to create a wire having an overall outer diameter close to 0.038″. Furthermore, a single wire may be encapsulated in a multi-strand braid.

The catheter chosen should be of soft material so that it may assume the shape of a tortuous vessel. Preferably, it should be free of any side holes, and the internal diameter should be chosen to closely mimic the internal diameter of the coil. Using a catheter of larger bore than the straightened length of the wire may cause the coil to curl within the passageway. The use of shape-memory wire allows the wire to have greater resiliency in bending, and thus permanent, plastic deformations may still be avoided even if difficulties are encountered during wire delivery.

Vessels with a serpentine configuration may complicate the coil delivery procedure. A vessel that is too tortuous may be inaccessible if standard catheters are employed. However, smaller catheters such as Tracker catheters may permit the vessel to be more easily negotiated, such as in cases of coronary AV fistulas. The advantage of such Tracker catheters is their ability to be tracked to the distal end of the fistula. The catheter is passed through larger guiding catheters which may be used to cannulate the feeding vessel such as the right or left coronary artery at its origin. Such a Tracker catheter may accommodate 0.018″ “micro-coils”.

Alternatively, in order to accommodate large coils such as 0.038″ coils, 4 F catheters such as those made by Microvena may be employed. For defects requiring such large coils, delivery may be made either from the arterial or venous end. Damage to the artery may be minimized if the femoral artery route is approached.

In patients requiring multiple coils, delivery may occur sequentially by accessing the duct in an alternating sequence from the arterial or venous route, or by simultaneous delivery from each route. In the latter case, the duct may be accessed by two or three catheters usually from the venous end. At least two coils may be released simultaneously in the aortic ampulla, with the pulmonary ends of the coils released sequentially. A third coil may be subsequently released through a third catheter placed at the duct. The advantage of the simultaneous technique is the ability to treat very large ducts with individual coil sizes that are less than two or three times the size of the duct. Both techniques may also be used in combination.

An example of multiple coil deployment is illustrative. In order to occlude a 5.7 mm duct, two 8 mm coils along with one 5 mm coil were deployed by the simultaneous technique as previously described. Subsequent to this deployment, three additional 5 mm coils were deployed using the sequential technique, in order to achieve complete occlusion. This combined use of deployment techniques was essential to the success of the procedure, since use of only the sequential approach in this case would have theoretically necessitated a coil approximately 12 to 16 mm in size. Such an extreme size may be particularly troublesome in young children, and may result in unacceptable blockage of the pulmonary artery or protrusion beyond the aortic ampulla. In addition, such a large coil might result in a high incidence of embolization of the first one or two coils.

In order to decrease the incidence of coil embolization, a controlled release coil is useful. Such a spring coil design, reminiscent of the Gianturco coil, may be provided with a central passageway through which a delivery mandril is passed. Interlocking screws between the spring coil and the delivery wire assist in securing the coil until it has been delivered to a proper position in the duct. The coil may then be released by unscrewing the locking device. The use of this controlled release technique has been attributed to a decrease from 9% to only 1.8% in the incidence of coil embolization.

In another preferred embodiment of the coil design, a plurality of active memory and passive memory elements are used. Advantageously, such a combination permits a desired coil stiffness and length to be achieved, and further facilitates the use of coils with extended ends or clips. In a preferred method of fabricating the coil, a coil wire is wound on top of a core wire using conventional winding techniques to create a multilayered wire. Preferably, a high precision winding device is used, such as the piezo-based winding system developed by Vandais Technologies Corporation of St. Paul, Minn. The coil wire is preferably rectangular or arcuate in cross-section, but other cross-sections such as a hexagonal shape or other polygonal shape may be used. The coil wire is also preferably substantially uniform in cross-section. However, a gradually tapered wire may also be used. Preferably, the dimensions of the layered coils are chosen such that comparatively thick sections formed from passive materials are avoided, due to expansion difficulties that may arise when the coils are warmed to their preset configuration. Subsequent to winding the coil wire/core wire combination, the multilayered wire is wound about a mandrel having a desired shape, preferably a shape permitting a final coil configured as shown in FIG. 1, 8 or 15. The coil may also be formed with or without clips for anchoring the device. The entire assembly is next transported to a furnace, wherein the multilayered wire is heat treated to set the desired shape. The temperature and duration of any heat treatment is a function of the materials used to form the multilayered wire. Following heat treatment, the assembly is removed from the furnace and allowed to cool to room temperature. The coil may then be removed from the mandrel. Depending on the materials used for the core wire and coil wire, a coil having a combination of active and passive memory elements may be produced.

In some alternate embodiments, the heat treating of the wire formed from a shape memory material is performed prior to winding a non-shape memory wire about it.

For example, nitinol coil wire may be used to confer active memory to the device, due to its shape memory and/or superelastic properties. Stainless steel, carbon fiber, or Kevlar® (poly-paraphenylene terephthalamide) fiber core wire may be used to confer passive memory because they are materials that may be given heat-set memory, but do not possess shape memory properties. Other appropriate passive-memory materials include relatively soft metals such as platinum and gold, relatively hard metals such as titanium or Elgiloy® (Cobalt-Chromium-Nickel alloy), or non-metals such as polytetrafluoroethylene (PTFE) or Dacron® (synthetic or natural fiber). The multilayered wire advantageously allows the device to possess several distinct materials properties; a wire layer of carbon fiber may allow an extremely flexible device shape, while a wire layer of nitinol may provide necessary rigidity. This combination enhances the ability of the device to retain its shape regardless of the type of defect or forces encountered during deployment and usage. Furthermore, the carbon fiber or other passive material facilitates the navigation of the device through tortuous anatomical regions.

If carbon fiber is used as the core wire, then the coil wire cannot be wound directly on the core. In such a case, a suitable mandril is first used to wind the coil wire, which is next subjected to a heat treatment in a furnace. After removal from the furnace and cooling, the mandril is removed and the carbon fiber is placed on the inner surface of the coil wire.

Alternatively, the madril may be removed after winding the coil wire, so that the core wire may be placed on the inner surface of the coil wire. The multilayered wire may then again be placed on the mandril, and subjected to a heat treatment to set the desired shape.

In an alternate embodiment, the coil wire is bordered by a core wire on the inner surface of the device, and an additional overlayer wire on the outer surface of the device. In yet another embodiment, the coil wire is provided as a twisted pair with the second wire of the pair being formed of either an active memory material or a passive memory material.

In yet another alternate embodiment of a coil and method of fabricating a coil having a combination of active memory and passive memory elements, a core wire is wound on top of a coil wire. The coil wire may serve as either the active or passive memory element. Likewise, the core wire may serve as either the active or passive memory element.

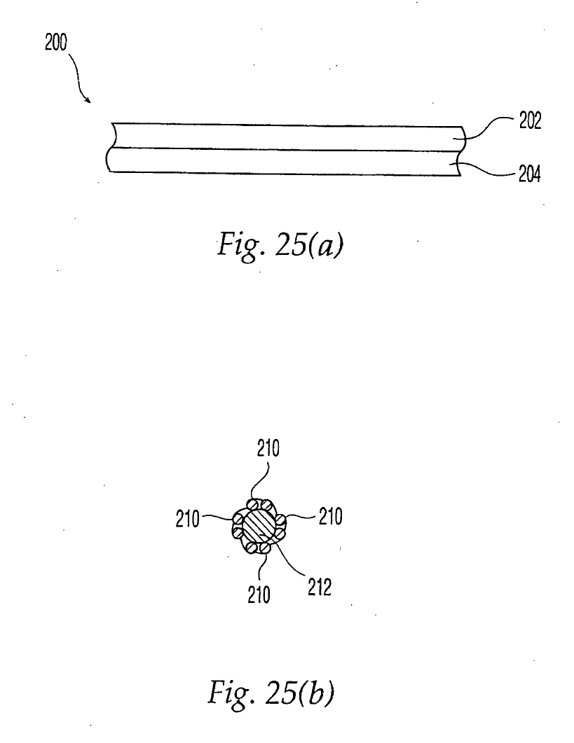

In addition, the core and coil wires may be disposed about each other in various configurations. The core wire, for example, may be disposed longitudinally about the coil wire (i.e., oriented in mirror-image fashion). For example, as shown in FIG. 25(a), a member 200 may be formed of layers 202, 204. Alternatively, the core wire may be wrapped about the coil wire in spiral fashion. If several core wires or several coil wires are to be used in combination, the wires may be disposed about each other using one or both of the longitudinal planking or radial wrapping orientations.

In a preferred embodiment, a capping process may also be undertaken to allow the ends of the core and the wire to be welded and capped in order to avoid any fraying.

In another preferred embodiment, a braid may also be wound on top of a central core. The braid may be wound to a desired pitch, with successive turns oriented extremely close together or at varying distances apart. For example, as shown in FIG. 25(b), braid portions 210 may be disposed around a central core 212. When braids are wound in spaced fashion, the mandril is left exposed at various intervals. After the madril is removed, a suitable intermediate material may be used in its place.

Various central core materials are contemplated, including plastic, metal, or even an encapsulated liquid or gel. In a preferred embodiment, an active memory/active memory combination is used, thus necessitating central cores and braids made of shape memory materials. In a most preferred embodiment, the central core and braid are both made of nitinol.

In an alternate embodiment, one of the central core and braid is an active memory element and the other is a passive memory element.

After the multilayered wire is wound on the core using a winding machine, the wound material may be released from the tension of the machine. If nitinol is used, the superelastic properties of the nitinol produce a tendency of the wound form to immediately lose its wound configuration. In order to retain the shape, an external mechanical or physical force may be applied, such as a plastic sleeve to constrain the material. If a plastic sleeve is used, it may be removed prior to heat treatment.

A multi-part mold may also be used. Due to the superelastic properties of nitinol wire, it may be necessary to further constrain the wire on the mandril during the manufacturing process. Thus, an inner mandril may be used for winding the wire to a desired shape. After winding, an outer mold may be used to completely surround the wire on the mandril to constrain its movement with respect to the mandril. The mandril and mold create a multi-part mold that may be transferred to a furnace for the heat treatment process. In a preferred heat treatment, the wire must be heated to a temperature of approximately 450-600° C. Depending on the material used to form the multi-part mold, the mold may need to be heated to a suitably higher temperature in order for the wire encased within the mold to reach its proper heat set temperature. Only a short heat treatment at the set temperature may be required, such as thirty minutes. After cooling, the device must be removed from the multi-part mold and carefully inspected for any surface or other defects.

In a preferred embodiment, the coil device is provided with at least one clip, located at the end of a loop. The clip allows the device to be anchored in the desired anatomical region of the body.

Due to the superelastic and shape memory properties of nitinol, various devices are contemplated. The superelastic properties allow the coils to have excellent flexibility, while the shape memory properties allow the coils to be delivered through conventional catheters that otherwise could not easily accommodate the diverse shapes.

As disclosed above, the present invention includes single coils 10, either used alone or in combination for occluding a duct. For large ducts, multiple coils may be required to occlude the duct. The multiple coils can be positioned within the duct either simultaneously, sequentially, or in combination of thereof. In such instances, it is contemplated that multiple coils 10 may be used to form a composite coil.

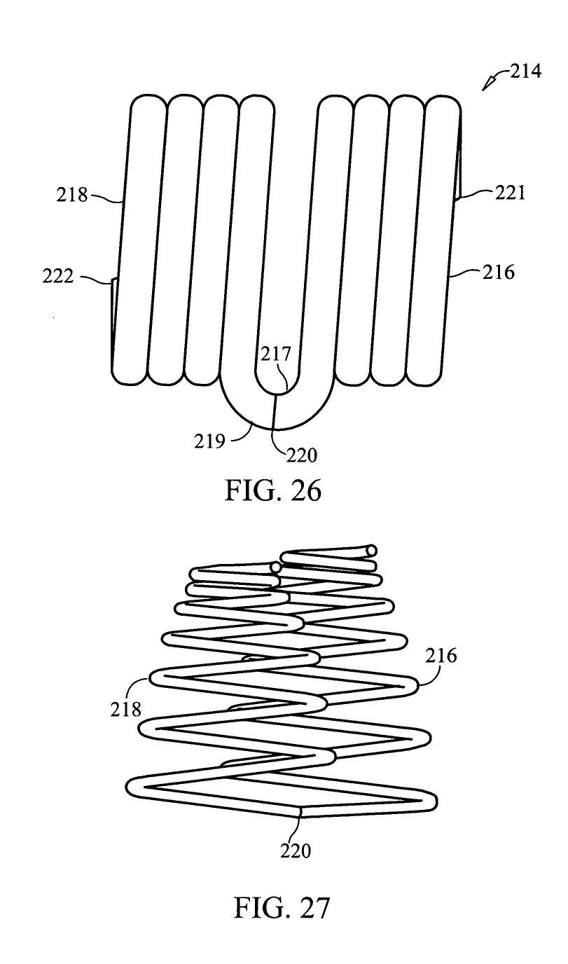

Referring to FIG. 26, a composite coil 214 includes at least a first and second coil 216 and 218 each including first ends 217 and 219 joined together at joint 220. The first and second coils 216 and 218 can be joined together such that the loops of the individual coils 216 and 218 are separate from or in the alternative, intertwined with each other (See FIG. 27). The coil first ends 217 and 219 can be joined by welding or other such bonding techniques. Each of the first and second coils 216 and 218 can take the form of one of the above disclosed coils 10. Alternatively, at least one of the coils 216 and 218 can be substantially linear.

As described above, each of the coils 216 and 218 may be adapted to optionally include a clip 223 on at least one of the coil second free ends 221 and 222. The clip 223 enhances attachment of the coil to its surroundings. The clip 223 may be a prong-like extension from the coil that has at least one generally straight section. Furthermore, the clip 223 may be oriented transverse to the central longitudinal axis of the coil 223, or it may extend parallel to the axis.

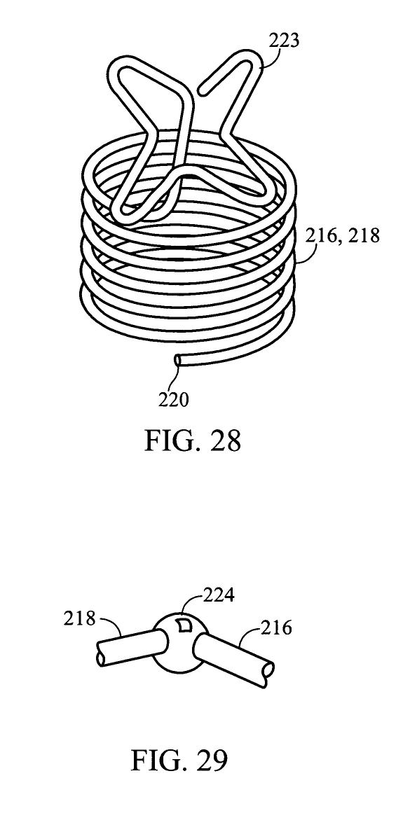

Referring to FIG. 28, the clip 223 may be in an fan or star-like configuration and may include at least two prongs for contacting the tissue at the desired location. The prongs may be curved prongs and/or sharp prongs. Advantageously, the use of prong configurations permits multiple anchor points to tissue, and thus also provides additional securing of the device. Alternatively, the clip 223 configuration may optionally be selected from the above described clips in FIG. 15

Each of the coils 216 and 218 in the composite coil 214 may have the same size, length, diameter, and/or configuration or have different sizes, lengths, diameters and/or configurations. The composite coil 214 provides the ability to treat very large ducts with a simultaneous insertion of multiple coils through a single cannula, wherein each of the individual coil sizes are less than two or three times the size of the duct. In one embodiment coil 216 is made of a material having first shape memory properties and coil 218 is made of a second material having second shape memory properties. The first shape memory properties differ from the second shape memory properties such that the occlusive behavior of coil 216 differs from that of coil 218.

As noted above, shape memory alloys may be deformed at a temperature below a transition temperature region that defines a region of phase change, and upon heating above the transition temperature region assumes an original shape. For example, NiTi alloys have two temperature-dependent phases, the martensite or lower temperature phase, and the austenite or higher temperature phase. When the alloy is in the martensitic phase, it may be deformed due to its soft, ductile, and even rubber-like behavior. In the austenitic phase, the alloy is much stronger and rigid, although still reasonably ductile, and has a significantly higher Young's Modulus and yield strength. While the material transforms from one phase to the other, the transformation temperature range is dependent on whether the material is being heated or cooled. The martensite to austenite transformation occurs during heating, beginning at an austenite start temperature, Aₛ, and ending at an austenite finish temperature, A_{f}. Similarly, the austenite to martensite transformation occurs during cooling, beginning at a martensite start temperature, Mₛ, and ending at a martensite finish temperature, M_{f}. Notably, the transition temperatures differ depending on heating and cooling, behavior known as hysteresis.

Some alloys display a “one-way” shape memory effect; essentially, this is an ability of the material to have a stored, fixed configuration (sometimes referred to as a trained shape), that may be deformed to a different configuration at a temperature below the phase change region, and subsequently may be heated above the transition temperature region to reassume that original configuration. A select group of alloys also display a “two-way” shape memory effect, in which the material has a first, fixed configuration at low temperature, and a second, fixed configuration at temperatures above the phase change. Thus, in this case, the material may be trained to have two different shapes.

Superelasticity (sometimes referred to as pseudoelasticity) occurs over a temperature range generally beginning at A_{f}, and ending when the NiTi is further heated to a martensite deformation temperature, M_{d}, that marks the highest temperature at which a stress-induced martensite occurs. In some cases, superelasticity may be observed at temperatures extending below A_{f}. The superelasticity of the material in this temperature range permits the material to be deformed without plastic deformation, and thus permanent deformation is avoided.

Referring to FIG. 29, a central hub member 224 can be used in the composite coil. The central hub member 224 is configured for receiving and coupling multiple coils 216 and 218. For example, the central hub member 224 can be spherical in shape, wherein at least one of each of the individual coils 216 and 218 is bonded to the surface of the central hub member 224. However, it is contemplated that the central hub member 224 can have other shapes, wherein the selected shape has sufficient surface area for receiving attachment of multiple coils thereto. The coils 216 and 218 can be bonded to the central hub member 224 by welding or other such bonding techniques.

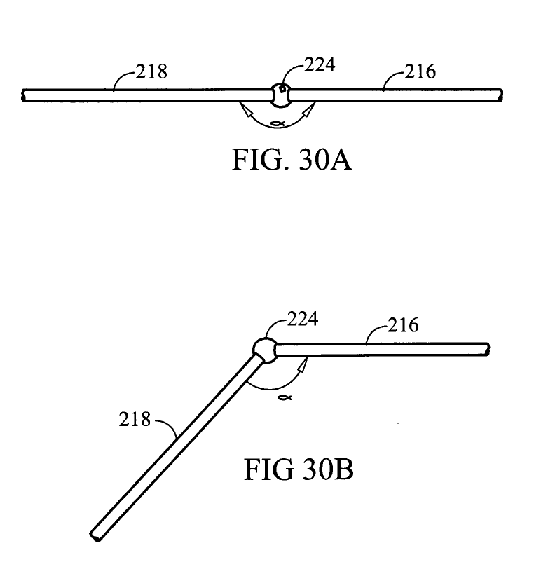

Referring to FIG. 30A, one or both of the coils 216 and 218 (or a substantial portion thereof) can be substantially linear, joined to the central hub member 224 at an angle α L of approximately 180° relative to each other. Alternatively, as shown in FIG. 30B the coils 216 and 218 can be joined to the central hub member 224 at an angle α less than 180° relative to each other.

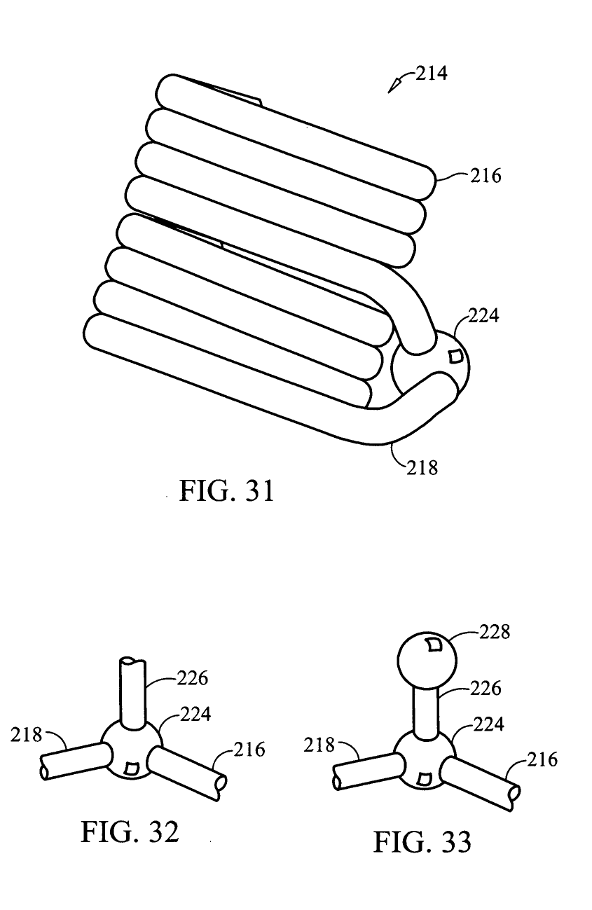

As shown in FIG. 31, coils 216 and 218 can be joined together such that the loops of the individual coils 216 and 218 are separate or in the alternative, intertwined with each other. The attachment position of the coils to the central hub is dependent on an number of factors, including by not limited to, the location and size of the duct and the size, shape, and dimension of the coils.

Additionally, as described above, there are several factors which are considered when choosing the size and shape of coils to be affixed to the central hub member 224 to be used in a particular application. The desired helical diameter of the coils, a measure of the final diameter of the coils after expansion to its circular shape and implantation, must be considered in light of the geometry. In addition, the length of the coils and the number of coil loops must be considered. Furthermore, coils may be designed with tightly packed windings, windings having only a short distance between each loop, or loosely packed windings having greater separation between neighboring loops. The length of the coils places an additional constraint on the number of loops that may be provided. Coils may be packaged and provided to the medical community based on any of the aforementioned factors, or a combination thereof.

Referring to FIG. 33, the central hub member 224 can include a neck portion 226 attached to and extending therefrom. The neck portion 226 is positioned on central hub member 224 such that it can be engaged by an insertion instrument for delivery into the body of the patient. For example, the neck portion 226 can be grasped by a bioptome, to aid the positioning of the composite coil 214 within a duct in the body of the patient.

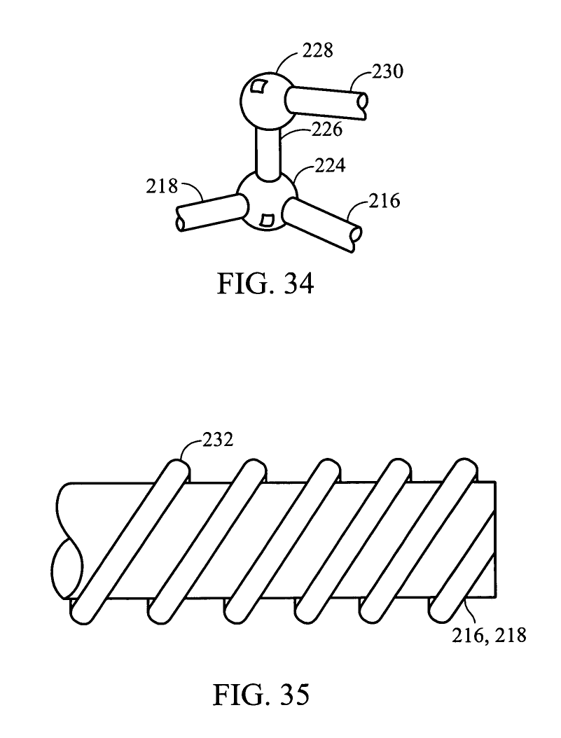

Referring to FIG. 33, the composite coil 214 further comprises a secondary hub member 228. The secondary hub member 228 is attached to the neck portion 226, opposite the central hub member 224. The secondary hub member 228 is sized to engage an insertion instrument, to aid in positioning the composite coil 214 in the body of the patient. Alternatively, as shown in FIG. 34, additional coils 230 can be attached to the secondary hub member 228.

Referring to FIG. 35, the coils 216 and 218 may be made more or less thrombogenic by attaching or weaving one or more fibers 232 along the length of the coils 216 and 218. For example active memory or passive memory fibers 232 are wound about the coils 216 and 218. When fibers 232 are wound in spaced fashion, the portion of the coils 216 and 218 are left exposed at various intervals. In an embodiment, Dacron strands are used.

As previously described, each component of the composite coil 214, including the individual coils 216 and 218, the central and secondary hub members 224 and 228, and the neck portion 226 may be made of a shape memory alloy. Such a material may be deformed at a temperature below a transition temperature region that defines a region of phase change, and upon heating above the transition temperature region assumes an original shape. The coil is preferably made of an alloy having shape-memory properties, including, but not limited to, the following alloys: Ni—Ti, Cu—Al—Ni, Cu—Zn, Cu—Zn—Al, Cu—Zn—Si, Cu—Sn, Cu—Zn—Sn, Ag—Cd, Au—Cd, Fe—Pt, Fe—Mn—Si, In—Ti, Ni—Al, and Mn—Cu. The coil is most preferably made of a nickel-titanium alloy. Such nickel-titanium alloys have gained acceptance in many medical applications, including stents used to reinforce vascular lumens. Additionally, the central and secondary hub members 224 and 228 and the neck portion may include active and/or passive memory elements.

Similar to single coils, the composite coil 214 may be delivered via a catheter that may be placed via a sheath. In its substantially straight configuration, the composite coil 214 should snugly fit in the catheter for slidable delivery.

The introduction mechanism of composite coil 214 may include a small tube that initially completely houses the straightened composite coil 214. The tube may be temporarily attached to the proximal end of a catheter, and the composite coil 214 may subsequently be inserted into the catheter with the help of a guidewire. The guidewire preferably is substantially straight, has a diameter similar to that of the wire used to form the coils 216 and 218, and additionally has a generally stiff end and a soft end. Once the composite coil 214 has been completely placed in the catheter, the tube is discarded, and the guidewire is used to place the composite coil 214 at the distal tip of the catheter and effect delivery of the device to the desired anatomical location.

In order to facilitate composite coil 214 delivery, radiopaque markers may be provided on the composite coil 214, either on the coils 216 and 218, central hub member 224, secondary hub member 228, or the neck 226. In an alternate embodiment, markers may be provided continuously or in spaced, regular intervals along the length of the coils 216 and 218. The use of such markers allows composite coil 214 delivery to be precisely monitored. Thus, if a composite coil 214 is not delivered properly to the chosen anatomical location, the composite coil 214 may be withdrawn into the sheath for re-release or may be completely withdrawn from the body.

As previously described, the present invention may be utilized as a filter, implantable in a blood vessel in the body of the patient. Such filters may utilize one or more members arranged to capture particulates within the blood flow, without substantially interfering with the normal blood flow.

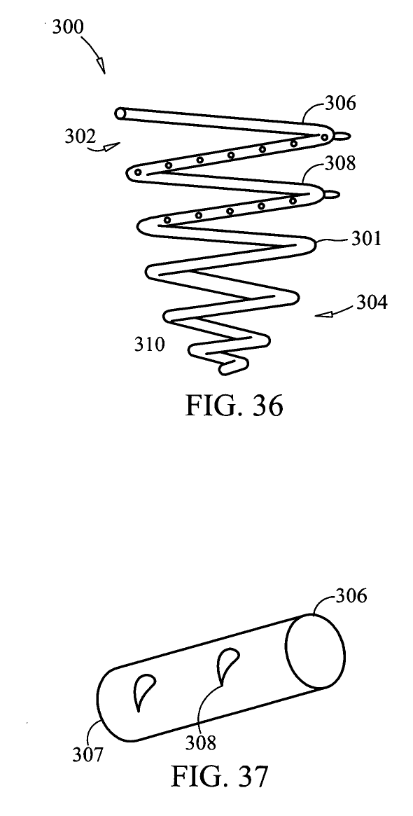

Referring to FIGS. 36 and 37, a filter 300 of the present invention includes a wire coil disposed about a longitudinal axis of the filter 300. The filter 300 can be made of a shape memory alloy, which when coiled has a first cylindrical portion 302 and a second conical portion 304. The loops 306 of the cylindrical portion 302 have a diameter of sufficient size to contact the inner walls of the vessel. The exterior surface 307 of the loops 306 of the cylindrical portion 302 include a plurality of barbs 308.

The conical portion 304 of the filter includes a series of loops 310 provided in a progressively decreasing diameter from one end of the conical portion 304 to the other. The loops 310 of the conical portion 304 can form a substantially conical coil having a constant or variable pitch. The loops 310 are provided in a spaced apart arrangement of a sufficient distance to capture particulates within the blood flow, without substantially interfering with the normal blood flow. The loop spacing can be dependent of the vessel diameter and the minimum particulate size, for example, the loops 310 can be spaced apart about 1.5-3 mm.

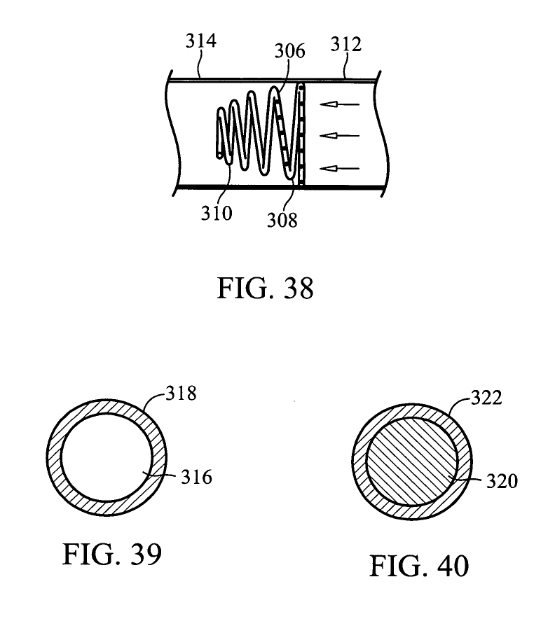

Referring to FIG. 38, the loops 306 of the cylindrical portion 302 provide a force against the inner wall 312 of the vessel 314, such that the barbs 308 are driven into the inner wall 312 of the vessel 314. The force of the loops 306 and the barbs 308 act together to anchor and stabilize the filter 300 within the vessel 314. The cylindrical portion 302 can include a plurality of loops 306, however, in a preferred embodiment, the cylindrical portion 302 includes two loops 306.

Referring to FIG. 39, the wire 316 of the filter 300 further includes an outer coating 318. The outer coating 318 can be bio-compatible, bio-neutral material which covers at least a portion of the filter 300. For example, the outer coating 318 can cover at least the cylindrical portion 302, substantially preventing adhesion of the tissue of the vessel 314 to the barbs 308 and exterior surface 307 of the cylindrical portion 302 of the filter 300. As such, the filter 300 can be removed without substantially tearing or damaging the inner wall 312 of the vessel 314. The outer coating 318 can additionally cover the cylindrical and conical portions 302 and 304 of the filter 300.

Alternatively, or in addition to, the wire 316 of the filter 300 may include an outer coating including a radio opaque material. The radio opaque material will make the filter 300 visible under fluoroscopy or X-ray imaging to aid in the placement of the filter 300 in the vessel 314.

Furthermore, the filter 300 can be coated with a drug or pharmaceutical agent. The drug can include an anti-restenotic drug which decreases or prevents encapsulation of the filter 300 with tissue growth. Exemplary anti-restenotic drugs include sirolimus and TAXOL®.

Similar to the previously described coils, filter 300 is preferably made of an alloy having shape-memory properties. The shape memory alloy can be made of a material having a one-way or two-way shape memory effect.

A “one-way” shape memory effect essentially is an ability of the material to have a stored, fixed configuration (sometimes referred to as a trained shape), that may be deformed to a different configuration at a temperature below the phase change region, and subsequently may be heated above the transition temperature region to reassume that original configuration. A “two-way” shape memory effect, is where the material has a first, fixed configuration at low temperature, and a second, fixed configuration at temperatures above the phase change. Thus, in this case, the material may be trained to have two different shapes.

The shape memory alloy can have temperature dependent material properties. These alloys have two temperature-dependent phases, the martensite or lower temperature phase, and the austenite or higher temperature phase. When the alloy is in the martensitic phase, it may be deformed due to its soft, ductile, and even rubber-like behavior. In the austenitic phase, the alloy is much stronger and rigid, although still reasonably ductile, and has a significantly higher Young's Modulus and yield strength. While the material transforms from one phase to the other, the transformation temperature range is dependent on whether the material is being heated or cooled. The martensite to austenite transformation occurs during heating, beginning at an austenite start temperature, Aₛ, and ending at an austenite finish temperature, A_{f}. Similarly, the austenite to martensite transformation occurs during cooling, beginning at a martensite start temperature, Mₛ, and ending at a martensite finish temperature, M_{f}. Notably, the transition temperatures differ depending on heating and cooling, behavior known as hysteresis.

In an embodiment, the shape memory alloy has an austenite finish temperature below body temperature, thereby permitting the filter 300 to have superelastic properties at body temperature.

The shape memory alloy can include, but not be limited to, the following alloys: Ni—Ti, Cu—Al—Ni, Cu—Zn, Cu—Zn—Al, Cu—Zn—Si, Cu—Sn, Cu—Zn—Sn, Ag—Cd, Au—Cd, Fe—Pt, Fe—Mn—Si, In—Ti, Ni—Al, and Mn—Cu. The filter 300 is most preferably made of a nickel-titanium alloy. Such nickel-titanium alloys have gained acceptance in many medical applications, including stents used to reinforce vascular lumens. Additionally, the filter 300 may include active and/or passive memory elements.

Referring to FIG. 40, the filter 300 may include a plurality of layers 320 and 322. At least one layer may be formed of a passive memory material, and in another embodiment at least two layers may be formed of active memory materials. A plurality of active memory and passive memory elements can be used, such that the combination permits a desired filter 300 stiffness.

Alternatively, the filter 300 can include several wires braided together in order to produce a braided wire with a desired outer diameter. Furthermore, a single wire may be encapsulated in a multi-strand braid. The braided wires can include a combination of active and passive elements, such that the combination of number braided wires and elements permits a desired filter 300 stiffness. At least one of the wires in the braid is made of a shape memory alloy.

The filter 300 can include a plurality of layers of braided wires. At least one braided layer may be formed of a passive memory material, and in another embodiment at least two braided layers may be formed of active memory materials. A plurality of active memory and passive memory elements can be used, such that the combination permits a desired filter 300 stiffness.

Alternatively, the filter 300 can include a plurality of layers, where at least one of the layers is a braided layer. At least one layer may be formed of a passive memory material, and in another embodiment at least two layers may be formed of active memory materials. A plurality of active memory and passive memory elements can be used, such that the combination permits a desired filter 300 stiffness.

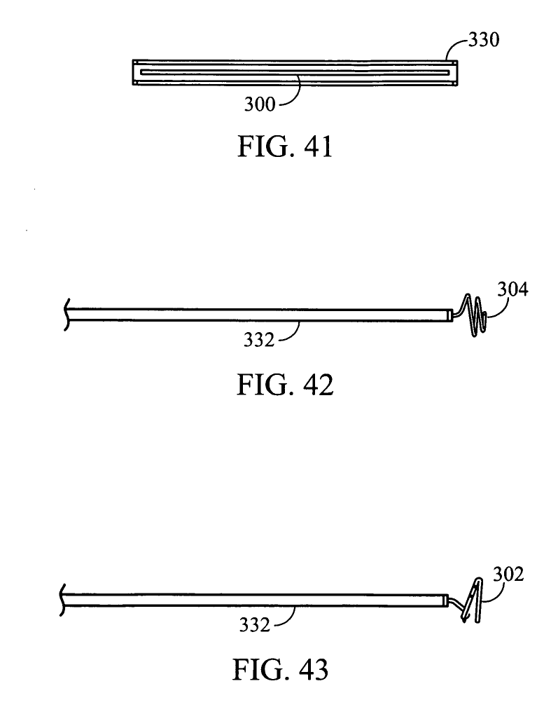

Referring to FIG. 41 the filter 300 can be provided within a cartridge 330 in a substantially linear configuration. To position the filter 300 in a vessel 314, the cartridge 330 is connected to an end position of a catheter (not shown), where the opposite end position of the catheter is positioned within the vessel 314. The filter 300, in the linear form, is then moved from the cartridge 330, through the catheter and into the vessel 314. Upon exiting the catheter, the filter 300 expands to the coiled configuration.

Depending on the method of insertion, via femoral approach or jugular approach, the cartridge 330 can be affixed to the catheter such that the filter 300 is appropriately oriented within the vessel 314. Referring to FIG. 42, the cartridge 330 is affixed to the catheter 332 such that the first portion of the filter 300 to exit the catheter 332 is the conical portion 304. Alternatively, referring to FIG. 43, the cartridge 330 is affixed to the catheter 332 such that the first portion of the filter 300 to exit the catheter 332 is the cylindrical portion 302.

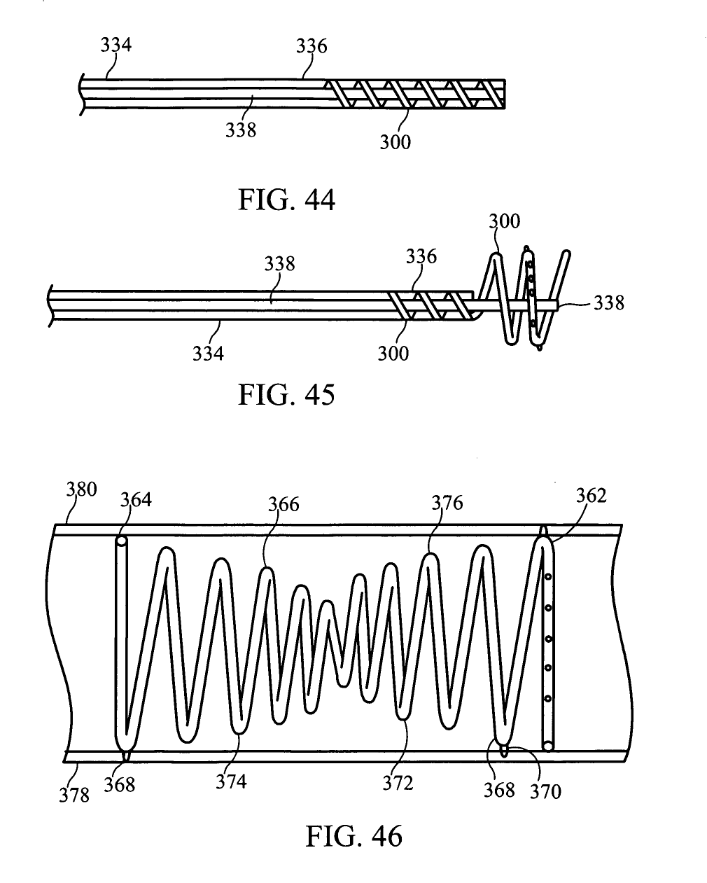

Referring to FIG. 44, in an alternative method of insertion, the filter 300 is provided within a catheter 334, wherein the catheter 334 includes a retractable end portion 336. The filter 300 is wrapped about a central guide 338, with the retractable end portion 336 positioned over the filter 300. The catheter 334 is inserted into the vessel 314, such that the retractable end portion 336 is positioned within the vessel 314. The retractable end portion 336 is retracted, exposing the filter 300 such that the filter 300 expands about the central guide 338. The retractable end portion 336 is retracted completely, exposing the filter 300 for placement in the vessel 314.

Depending on the method of insertion, via femoral approach or jugular approach, the filter 300 is positioned about the central guide 338 such that the filter 300 is appropriately oriented within the vessel 314. The filter 300 can be positioned about the central guide 338 such that the first portion expanded about the central guide 338 is the conical portion 304. Alternatively, the filter 300 can be positioned about the central guide 338 such that the first portion expanded about the central guide 338 is the cylindrical portion 302.

In an embodiment, the filter 300 of the present invention is a vena cava filter. The vena cava filter 300 is implantable in the inferior vena cava, and is utilized to filter peripheral venous blood clots. The filter 300 can be permanently or removably implanted.

Referring to FIG. 46, a filter 360 of the present invention includes a wire coil disposed about a longitudinal axis of the filter 360. The filter 360 can be made of a shape memory alloy, which when coiled has first and second cylindrical portions 362 and 364 and a narrowed section 366 interposed therebetween. The loops 368 of the cylindrical portions 362 and 364 have a diameter of sufficient size to contact the inner walls of the vessel. The exterior surface of the loops 368 of the cylindrical portions 362 and 364 include a plurality of barbs 370 (see also FIG. 37).

The narrowed section 366 includes a pair of opposing conical portions 372 and 374, which each include a series of loops 376 provided in a progressively decreasing diameter from one end of the conical portions 372 and 374 to the other. The loops 376 of the conical portions 372 and 374 can form a substantially conical coil having a constant or variable pitch. The loops 376 can be provided in a spaced apart arrangement of a sufficient distance to capture particulates within the blood flow, without substantially interfering with the normal blood flow.

The loops 368 of the cylindrical portions 362 and 364 provide a force against the inner wall 378 of the vessel 380, such that the barbs 370 are driven into the inner wall 378 of the vessel 380. The force of the loops 368 and the barbs 370 act together to anchor and stabilize the filter 360 within the vessel 380.

Similar to the above described filter 300, the wire of the filter 360 further includes an outer coating. The outer coating can be bio-compatible, bio-neutral material which covers at least a portion of the filter 360. The outer coating can substantially prevent adhesion of the tissue of the vessel 380 to the filter 360. As such, the filter 360 can be removed without substantially tearing or damaging the vessel 380.

Furthermore, the filter 360 can be coated with a drug or pharmaceutical agent. The drug can include and anti-restenotic drug which decreases or prevents encapsulation of the filter 360 with tissue growth. Exemplary anti-restenotic drugs include sirolimus and TAXOL®. Additionally, a drug can be provided which promotes the healing of the repaired area.

The filter 360 is preferably made of an alloy having shape-memory properties. The shape memory alloy can be made of a material having a one-way or two-way shape memory effect. Additionally, the shape memory alloy can have temperature dependent material properties.

The filter 360 may include a plurality of layers. At least one layer may be formed of a passive memory material, and in another embodiment at least two layers may be formed of active memory materials. A plurality of active memory and passive memory elements can be used, such that the combination permits a desired stiffness.

The wire can include several wires braided together in order to produce a braided wire with a desired outer diameter. Furthermore, a single wire may be encapsulated in a multi-strand braid. The braided wires can include a combination of active and passive elements, such that the combination of number braided wires and elements permits a desired stiffness. At least one of the wires in the braid is made of a shape memory alloy.

The filter 360 can include a plurality of layers of braided wires. At least one braided layer may be formed of a passive memory material, and in another embodiment at least two braided layers may be formed of active memory materials. A plurality of active memory and passive memory elements can be used, such that the combination permits a desired stiffness.

Alternatively, filter 360 can include a plurality of layers, where at least one of the layers is a braided layer. At least one layer may be formed of a passive memory material, and in another embodiment at least two layers may be formed of active memory materials. A plurality of active memory and passive memory elements can be used, such that the combination permits a desired stiffness.

The filter 360 can be inserted similarly to filter 300 as shown in FIGS. 41 and 44. Referring to FIG. 41 the filter 360 can be provided within a cartridge 330 in a substantially linear configuration. To position the filter 360 in a vessel 314, the cartridge 330 is connected to an end position of a catheter (not shown), where the opposite end position of the catheter is positioned within the vessel 314. The filter 360, in the linear form, is then moved from the cartridge 330, through the catheter and into the vessel 314. Upon exiting the catheter, the filter 360 expands to the coiled configuration.

Referring to FIG. 44, in an alternative method of insertion, the filter 360 is provided within a catheter 334, wherein the catheter 334 includes a retractable end portion 336. The filter 360 is wrapped about a central guide 338, with the retractable end portion 336 positioned over the filter 360. The catheter 334 is inserted into the vessel 314, such that the retractable end portion 336 is positioned within the vessel 314. The retractable end portion 336 is retracted, exposing the filter 360 such that the filter 360 expands about the central guide 338. The retractable end portion 336 is retracted completely, exposing the filter 360 for placement in the vessel 314.

In an embodiment, the filter 360 of the present invention is a vena cava filter. The vena cava filter 360 is implantable in the inferior vena cava, and is utilized to filter peripheral venous blood clots. The filter 300 can be permanently or removably implanted.

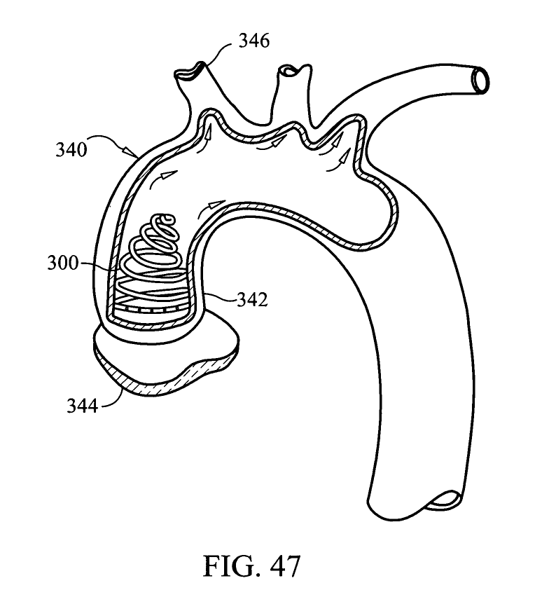

Referring to FIG. 47, in an embodiment, the filter 300 is positioned in the aortic arch 340 of the aorta providing cerebral embolic protection. The filter 300 is positioned in the base 342 of the aortic arch 340, between the aortic valve 344 and the brachiocephalic artery 346. Any potential emboli are captured by the filter, thereby preventing entry into the neurovasculature.

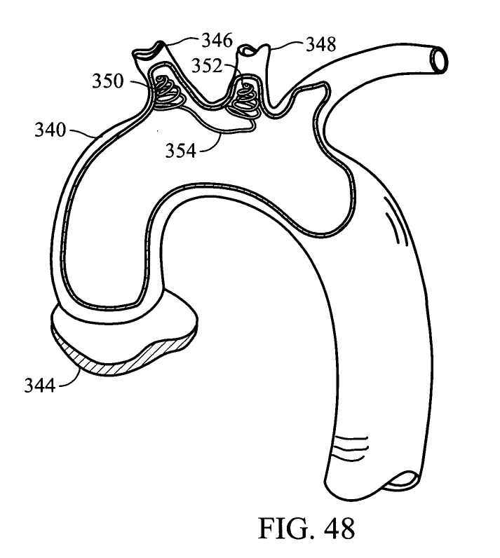

Referring to FIG. 48, in an embodiment, a first filter 350 is positioned in the brachiocephalic artery 346 and a second filter 352 is positioned in the left common carotid artery 348 of the aortic arch 340. Any potential emboli are captured by the filters 350 and 352, thereby preventing entry into the neurovasculature. The filters 350 and 352 can be permanently or removably implanted. A tether 354 can be provided, where the tether 354 connects the first and second filters 350 and 352. Tether 354 can be useful for insertion and/or removal of first and second filters 350 and 352. Tether 354 can be made of metallic material (like the filters) a polymeric material, or composite. In one embodiment, tether 354 has elastic behavior through a range of expansion. This elastic behavior is useful for accommodating different anatomies.

In a further embodiment, the present invention may be utilized as anatomic junction or bridge. An anatomic junction can be used in the repair of damaged or grafted vessels.

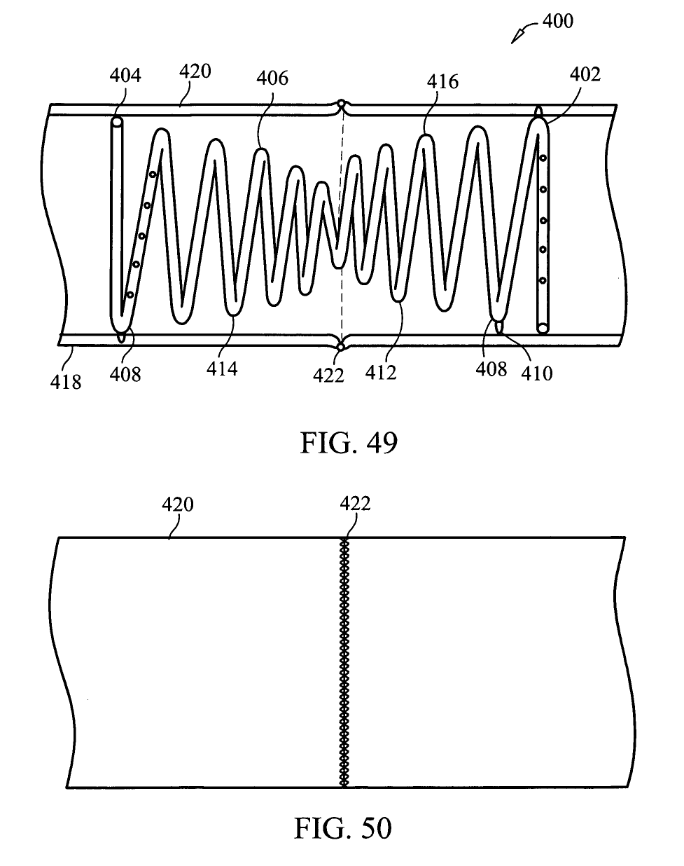

Referring to FIGS. 49 and 50, an anatomic junction 400 of the present invention includes a wire coil disposed about a longitudinal axis of the anatomic junction 400. The anatomic junction 400 can be made of a shape memory alloy, which when coiled has first and second cylindrical portions 402 and 404 and a narrowed section 406 interposed therebetween. The loops 408 of the cylindrical portions 402 and 404 have a diameter of sufficient size to contact the inner walls of the vessel. The exterior surface of the loops 408 of the cylindrical portions 402 and 404 include a plurality of barbs 410 (see also FIG. 37).

The narrowed section 406 includes a pair of opposing conical portions 412 and 414, which each include a series of loops 416 provided in a progressively decreasing diameter from one end of the conical portions 412 and 414 to the other. The loops 416 of the conical portions 412 and 414 can form a substantially conical coil having a constant or variable pitch. The loops 416 can be provided in a spaced apart arrangement of a sufficient distance to capture particulates within the blood flow, without substantially interfering with the normal blood flow.

The loops 408 of the cylindrical portions 402 and 404 provide a force against the inner wall 418 of the vessel 420, such that the barbs 410 are driven into the inner wall 418 of the vessel 420. The force of the loops 408 and the barbs 410 act together to anchor and stabilize the anatomic junction 400 within the vessel 420.

The anatomic junction 400 is positioned in the vessel 420, such that a sutured section 422 of the vessel 420 is interposed between the cylindrical portions 402 and 404 of the anatomic junction 400, about the narrowed section 406. The anatomic junction 404 can provide additional strength and stability to the sutured section 422 of the vessel 420, substantially preventing a tearing or separation.

Similar to the above described filter 300, the wire of the anatomic junction 400 further includes an outer coating. The outer coating can be bio-compatible, bio-neutral material which covers at least a portion of the anatomic junction 400. The outer coating can substantially prevent adhesion of the tissue of the vessel 420 to the anatomic junction 400. As such, the anatomic junction 400 can be removed without substantially tearing or damaging the repaired vessel 420.

Furthermore, the anatomic junction 400 can be coated with a drug or pharmaceutical agent. The drug can include and anti-restenotic drug which decreases or prevents encapsulation of the anatomic junction 400 with tissue growth. Exemplary anti-restenotic drugs include sirolimus and TAXOL®. Additionally, a drug can be provided which promotes the heal of the repaired area.

The anatomic junction 400 is preferably made of an alloy having shape-memory properties. The shape memory alloy can be made of a material having a one-way or two-way shape memory effect. Additionally, the shape memory alloy can have temperature dependent material properties.

The anatomic junction 400 may include a plurality of layers. At least one layer may be formed of a passive memory material, and in another embodiment at least two layers may be formed of active memory materials. A plurality of active memory and passive memory elements can be used, such that the combination permits a desired stiffness.

The wire can include several wires braided together in order to produce a braided wire with a desired outer diameter. Furthermore, a single wire may be encapsulated in a multi-strand braid. The braided wires can include a combination of active and passive elements, such that the combination of number braided wires and elements permits a desired stiffness. At least one of the wires in the braid is made of a shape memory alloy.

The anatomic junction 400 can include a plurality of layers of braided wires. At least one braided layer may be formed of a passive memory material, and in another embodiment at least two braided layers may be formed of active memory materials. A plurality of active memory and passive memory elements can be used, such that the combination permits a desired stiffness.

Alternatively, the anatomic junction 400 can include a plurality of layers, where at least one of the layers is a braided layer. At least one layer may be formed of a passive memory material, and in another embodiment at least two layers may be formed of active memory materials. A plurality of active memory and passive memory elements can be used, such that the combination permits a desired stiffness.

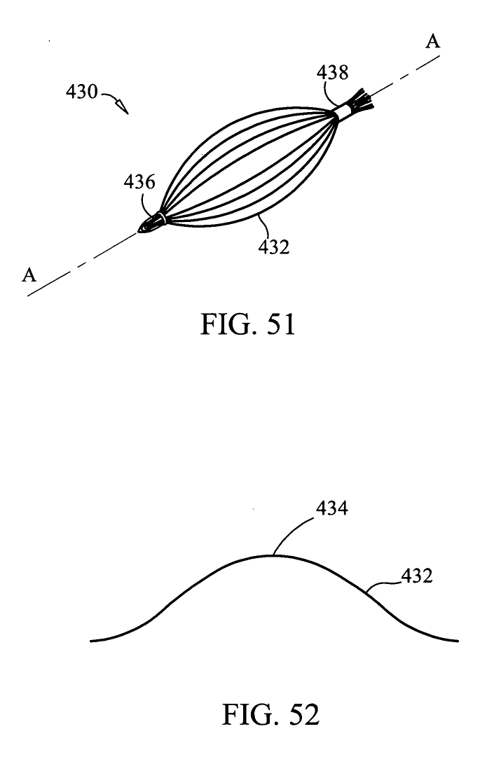

Referring to FIGS. 51 and 52, a filter 430 of the present invention includes a plurality of wire forms 432 circumferentially disposed about a longitudinal axis “A” of the filter 430. The filter 430 can be made of a shape memory alloy, wherein each of the wire forms 432 are provided in a curved-shape. The curved portions 434 of the wire forms 432 have a radius of sufficient size to contact the inner walls of the vessel.

The wire forms 432 are circumferentially positioned about the longitudinal axis “A” and first and second ends 436 and 438 are crimped, twisted, or welded together such that the filter 430 retains its shape. The wire forms 432 can be provided in a spaced apart arrangement of a sufficient distance to capture particulates within the blood flow, without substantially interfering with the normal blood flow.

The curved portion 434 of wire forms 452 provide a force against the inner wall of the vessel, such that an outward pressure and frictional force are exerted on the inner wall to anchor and stabilize the filter 430 within the vessel.

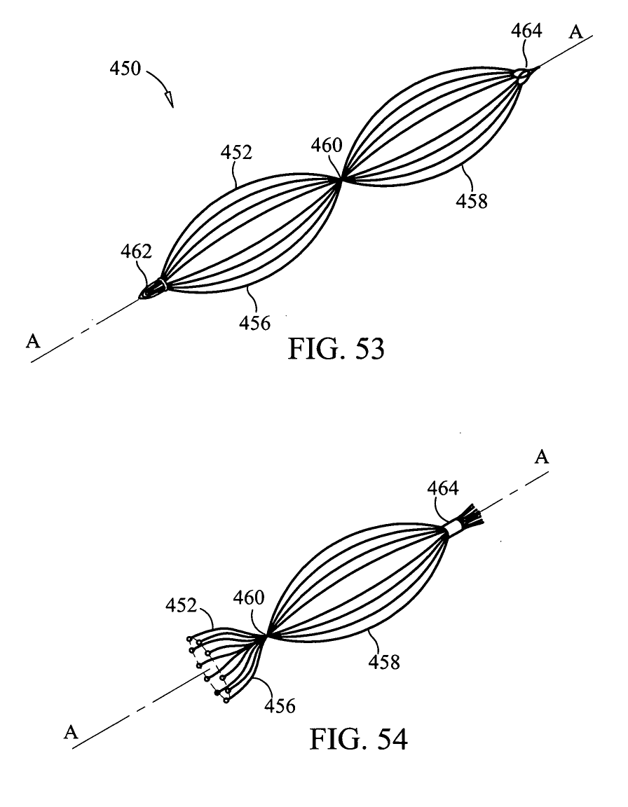

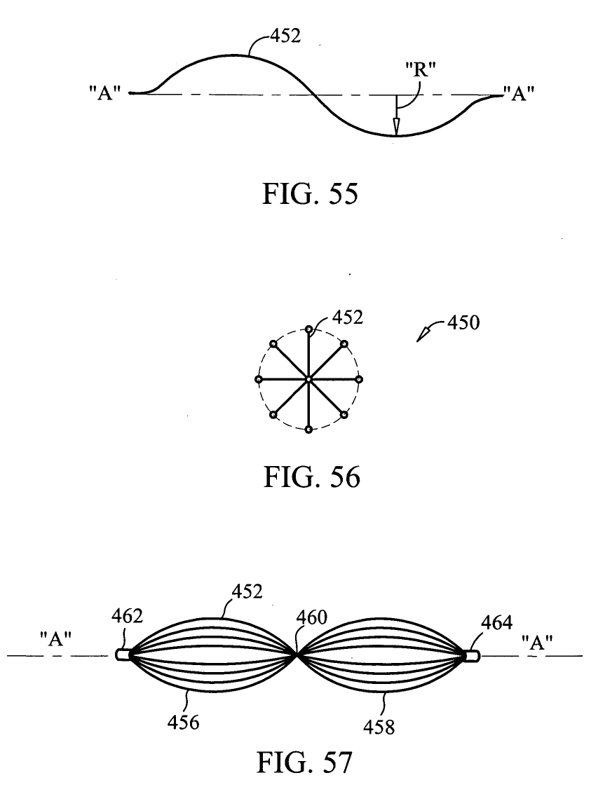

Referring to FIGS. 53-57, a filter 450 of the present invention includes a plurality of wire forms 452 circumferentially disposed about a longitudinal axis “A” of the filter 450. The filter 450 can be made of a shape memory alloy, wherein each of the wire forms 452 is provided in a substantially S-shape. The curved portions 454 of the S-shape of the wire forms 452 have a radius of sufficient size to contact the inner walls of the vessel.

The wire forms 452 are circumferentially positioned about the longitudinal axis “A” such that first and second sections 456 and 458 are formed and have a narrowed section 460 interposed therebetween. The wire forms 452 are crimped or twisted together at first and second ends 462 and 464 and intertwined about the narrowed section 460, such that the filter 450 retains its shape. The wire forms 452 can be provided in a spaced apart arrangement of a sufficient distance to capture particulates within the blood flow, without substantially interfering with the normal blood flow.

The first and second sections 456 and 458 of wire forms 452 provide a force against the inner wall of the vessel, such that an outward pressure and frictional force are exerted on the inner wall to anchor and stabilize the filter 450 within the vessel.

The filter 450 is disclosed as having wire forms 452 with two curved portion 454, in a substantially s-shape, forming first and second sections 456 and 458. However, it is contemplated that the wire forms 452 can have more than two curved portions, forming a plurality of sections disposed along the longitudinal axis “A.”

Similar to the above described filters, the wire of the filters 430 and 450 can further include an outer coating. The outer coating can be bio-compatible, bio-neutral material which covers at least a portion of the filters 430 and 450. The outer coating can substantially prevent adhesion of the tissue of the vessel to the filters 430 and 450. As such, the filters 430 and 450 can be removed without substantially tearing or damaging the repaired vessel.

Furthermore, the filters 430 and 450 can be coated with a drug or pharmaceutical agent. The drug can include and anti-restenotic drug which decreases or prevents encapsulation of the filters 430 and 450 with tissue growth. Exemplary anti-restenotic drugs include sirolimus and TAXOL®. Additionally, a drug can be provided which promotes the healing of the repaired area. The drug can be provided directly on the wire forms or incorporated in a polymer matrix.

The filters 430 and 450 are preferably made of an alloy having shape-memory properties. The shape memory alloy can be made of a material having a one-way or two-way shape memory effect. Additionally, the shape memory alloy can have temperature dependent material properties.

The filters 430 and 450 may include a plurality of layers. At least one layer may be formed of a passive memory material, and in another embodiment at least two layers may be formed of active memory materials. A plurality of active memory and passive memory elements can be used, such that the combination permits a desired stiffness.

The wire can include several wires braided together in order to produce a braided wire with a desired outer diameter. Furthermore, a single wire may be encapsulated in a multi-strand braid. The braided wires can include a combination of active and passive elements, such that the combination of number braided wires and elements permits a desired stiffness. At least one of the wires in the braid is made of a shape memory alloy.

The wire forms 432 and 452 can include a plurality of layers of braided wires. At least one braided layer may be formed of a passive memory material, and in another embodiment at least two braided layers may be formed of active memory materials. A plurality of active memory and passive memory elements can be used, such that the combination permits a desired stiffness.

Alternatively, wire forms 432 and 452 can include a plurality of layers, where at least one of the layers is a braided layer. At least one layer may be formed of a passive memory material, and in another embodiment at least two layers may be formed of active memory materials. A plurality of active memory and passive memory elements can be used, such that the combination permits a desired stiffness.

The filters 430 and 450 can be inserted into the vessel through a catheter or other similar type device.

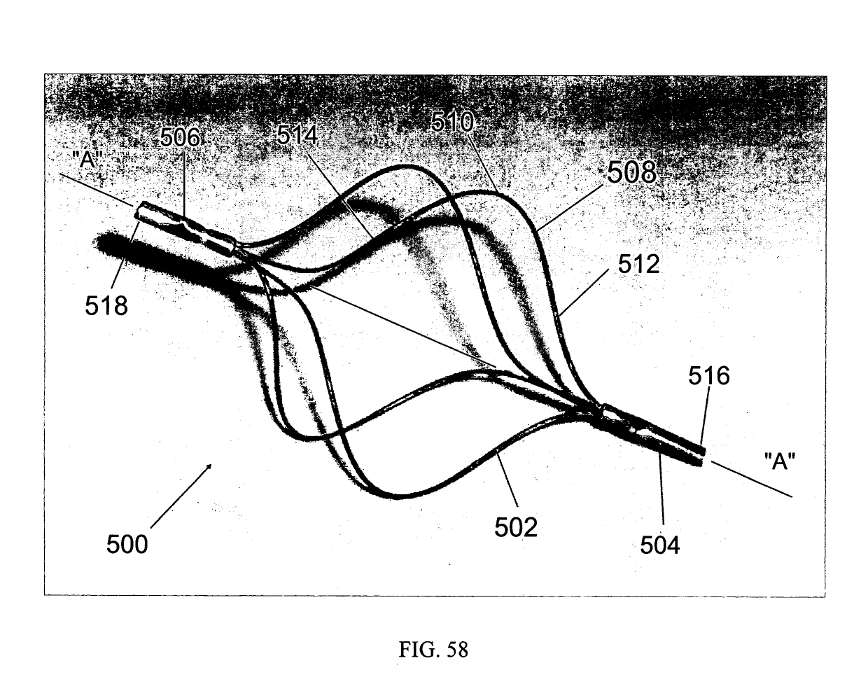

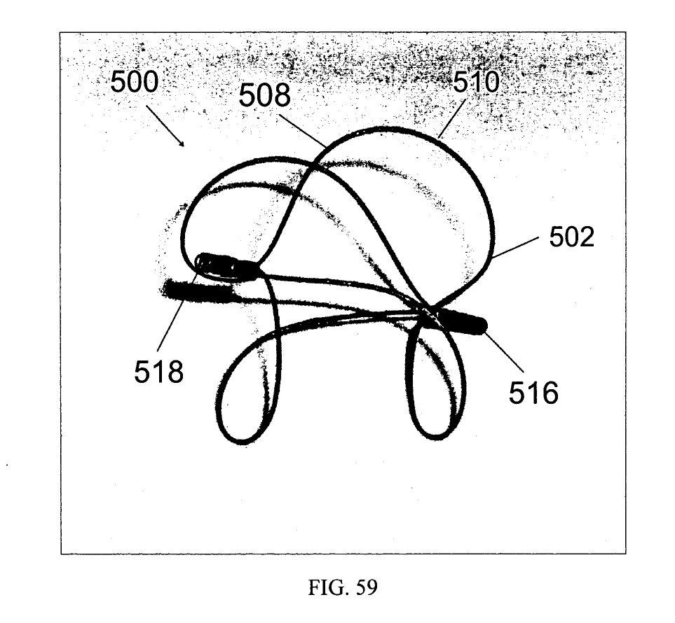

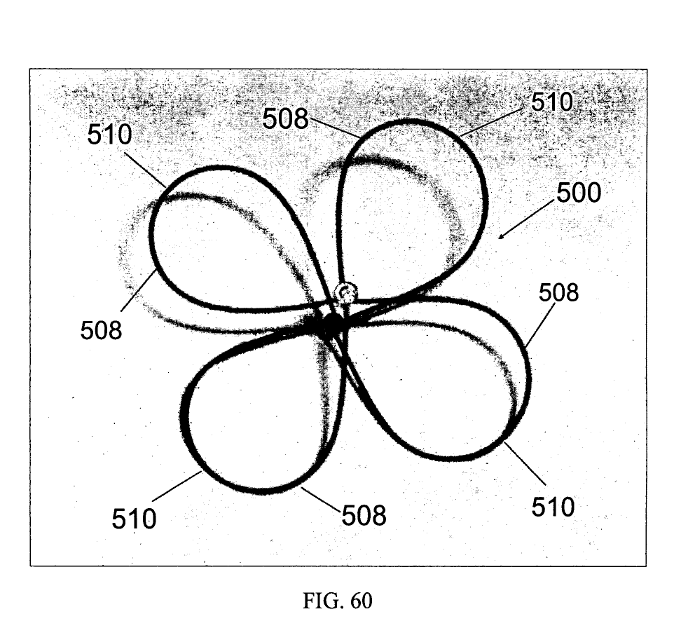

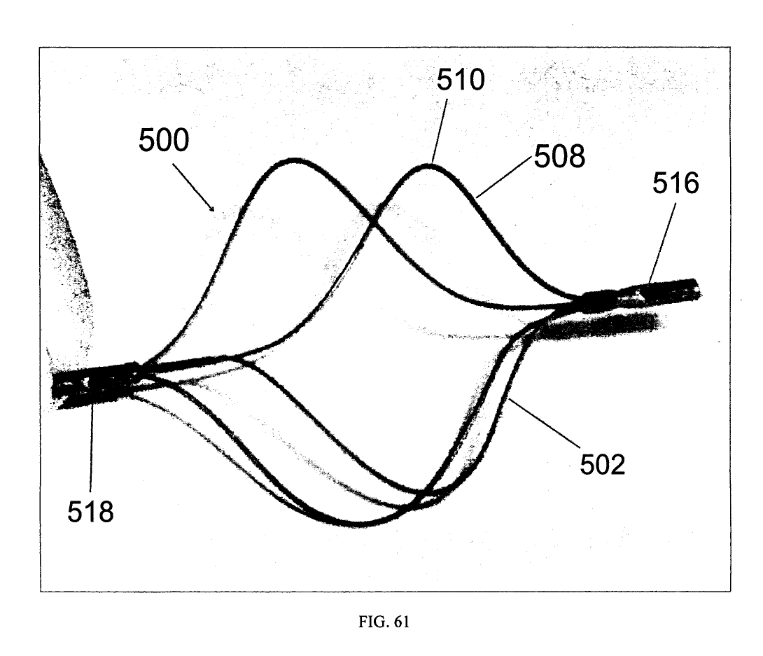

Referring to FIGS. 58-61, a filter 500 of the present invention includes a plurality of wire forms 502 circumferentially disposed about a central longitudinal axis “A” of the filter 500. Each of the wire forms 502 includes first and second end portions 504 and 506, where a curved portion 508 is interposed between the first end second end portions 504 and 506. The curved portion 508 is formed along the wire form 502, whereby the wire form 502 is initiated at the first end portion 504, along the central longitudinal axis “A,” and extends radially outward in a substantially axial and circumferential direction from and about the central longitudinal axis “A”, to a maximum diameter section 510. From the maximum diameter section 510, the wire form 502 extends radially inward in a substantially axial and circumferential direction to and about the central longitudinal axis “A,” terminating at the second end portion 506, along the central longitudinal axis “A.” In this manner, the wire form 502 is radially twisted about the central longitudinal axis “A.”

In an exemplary embodiment, the curved portion 508 is formed along the wire form 502, whereby the wire form 502 is initiated at the first end portion 504, along the central longitudinal axis “A,” and extends radially outward 512 along the central longitudinal axis “A” to the curved portion 508. The curved portion 508 extends in substantially axial and circumferential direction from and about the central longitudinal axis “A,” having a maximum diameter section 510. From the curved portion 508, the wire form 502 extends radially inward 514 along the central longitudinal axis “A,” terminating at the second end portion 506. In this manner, the curved portion 508 of the wire form 502 is radially spaced from and twisted about the central longitudinal axis “A.”

The filter 500 is formed by positioning a plurality of the wire forms 502 about the central longitudinal axis “A,” whereby the first and second end portions 504 and 506 of the wire forms 502 are affixed together, forming the first and second filter ends 516 and 518. The first and second end portions 504 and 506 of the wire forms 502 can be affixed together by twisting, crimping, or welding. The wire forms 502 are positioned about the central longitudinal axis “A” in a staggered arrangement, such that the maximum diameter section 510 of adjacent wire forms 502 are positioned at different axial distances from the first and second filter ends 516 and 518.

The maximum diameter section 510 of each of the wire forms 502 is located at about the same radial distance from the central longitudinal central axis “A.” The radial distance of the maximum diameter section 510 is selected, such that the maximum diameter sections 510 provide a force against the inner wall of the vessel, whereby an outward pressure and frictional force are exerted on the inner wall to anchor and stabilize the filter 500 within the vessel.

The number of wire forms 502 included in the filter 500 is dependent on the vessel diameter and the size of the particles to be captured, with the wire forms 502 provided in a spaced apart arrangement of a sufficient distance to capture particulates within the blood flow, without substantially interfering with the normal blood flow. For example, the filter 500 can include four, five, or six wire forms 502.

The filter 500 is disclosed as having wire forms 502 with single curved portion 508 in a substantially twisted shape. However, it is contemplated that the wire forms 502 can have two or mores curved portions, forming a plurality of filter sections disposed along the central longitudinal axis “A.”

Similar to the above described filters, the wire forms 502 of the filter 500 can further include an outer coating. The outer coating can be bio-compatible, bio-neutral material which covers at least a portion of the wire forms 502. The outer coating can substantially prevent adhesion of the tissue of the vessel to the wire forms 502. For example, the outer coating can be a polymeric coating. As such, the filter 500 can be removed without substantially tearing or damaging the repaired vessel.

Furthermore, the wire forms 502 of the filter 500 can be coated with a drug or pharmaceutical agent. The drug can include and anti-restenotic drug which decreases or prevents encapsulation of the filter 500 with tissue growth. Exemplary anti-restenotic drugs include sirolimus and TAXOL®. Additionally, a drug can be provided which promotes the healing of the repaired area. The agent can be coated directly onto the filter 500 or can be part of a polymeric matrix.

The wire forms 502 of the filter 500 are preferably made of an alloy having shape-memory properties. The shape memory alloy can be made of a material having a one-way or two-way shape memory effect. Additionally, the shape memory alloy can have temperature dependent material properties.

The wire forms 502 of filter 500 may include a plurality of layers. At least one layer may be formed of a passive memory material, and in another embodiment at least two layers may be formed of active memory materials. A plurality of active memory and passive memory elements can be used, such that the combination permits a desired stiffness.

The wire forms 502 can include several wires braided together in order to produce a braided wire with a desired outer diameter. Furthermore, a single wire may be encapsulated in a multi-strand braid. The braided wires can include a combination of active and passive elements, such that the combination of number braided wires and elements permits a desired stiffness. At least one of the wires in the braid is made of a shape memory alloy.

The wire form 502 can include a plurality of layers of braided wires. At least one braided layer may be formed of a passive memory material, and in another embodiment at least two braided layers may be formed of active memory materials. A plurality of active memory and passive memory elements can be used, such that the combination permits a desired stiffness.

Alternatively, wire forms 502 can include a plurality of layers, where at least one of the layers is a braided layer. At least one layer may be formed of a passive memory material, and in another embodiment at least two layers may be formed of active memory materials. A plurality of active memory and passive memory elements can be used, such that the combination permits a desired stiffness.

In a method of manufacture, the wire forms 502 are heat set in the twisted shape. The wire forms 502 are then coated/jacketed with the bio-compatible, bio-neutral material. The coated wire forms 502 are circumferentially positioned about the central longitudinal axis “A,” with the ends 504 and 506 of the wire forms 502 crimped together forming the filter 500.

The filter 500 can be inserted into the vessel through a catheter or other similar type device in a compressed or flattened form, where the filter 500 expands in the vessel, such that the maximum diameter 510 of the curved portions 508 stabilize and secure the position of the filter 500 within the vessel. Such a compressed or flattened form can be achieved by pulling apart, increasing the axial distance between, the filter ends 516 and 518. In this manner, the maximum diameter sections 510 of each of the wire forms 502 is drawn radially toward the central longitudinal axis “A.” Upon insertion, the material properties of the wire forms 502 expand the filter 500, drawing together, decreasing the axial distance between, the filter ends 516 and 518. In this manner, the maximum diameter sections 510 of each of the wire forms 502 is radially expanded toward the vessel wall. It is contemplated that the filter 500 can be inserted either through a femoral or jugular approach as previously described.

All references cited herein are expressly incorporated by reference in their entirety.

While various descriptions of the present invention are described above, it should be understood that the various features may be used singly or in any combination thereof. Therefore, this invention is not to be limited to only the specifically preferred embodiments depicted herein.

Further, it should be understood that variations and modifications within the spirit and scope of the invention may occur to those skilled in the art to which the invention pertains. Accordingly, all expedient modifications readily attainable by one versed in the art from the disclosure set forth herein that are within the scope and spirit of the present invention are to be included as further embodiments of the present invention. The scope of the present invention is accordingly defined as set forth in the appended claims.

1.-23. (canceled)

24. A vascular filter comprising:
a plurality of shaped wire forms circumferentially disposed about a longitudinal axis and each lying in a plane along the longitudinal axis, each of the plurality of shaped wire forms having a curved section, and first and second ends, wherein the first end of each of wire forms are affixed together and the second ends of each of the wire forms are affixed together.

25. A vascular filter as set forth in claim 24, wherein each of the plurality of wire forms consists of a single curved section.

26. A vascular filter as set forth in claim 24, wherein each of the plurality of wire forms includes a plurality of planar curved sections linearly disposed along the longitudinal axis.

27. A vascular filter as set forth in claim 24, wherein the first and second ends of the plurality of wire forms are affixed together by crimping, twisting, or welding.

28. A vascular filter as set forth in claim 24, further comprising a biocompatible coating covering at least a portion of the wire forms.

29. A vascular filter as set forth in claim 24, wherein the wire forms are formed of a shape memory alloy.

30. A vascular filter as set forth in claim 29, wherein the shape memory alloy displays a one-way shape memory effect.

31. A vascular filter as set forth in claim 29, wherein the shape memory alloy displays a two-way shape memory effect.

32. A vascular filter as set forth in claim 29, wherein the shape memory alloy has an austenite finish temperature below body temperature, thereby permitting the wire forms to have superelastic properties at body temperature.

33. A vascular filter as set forth in claim 29, wherein the shape memory alloy member includes a plurality of layers.

34. A vascular filter as set forth in claim 33, wherein the plurality of layers includes at least one layer formed of a passive memory material.

35. A vascular filter as set forth in claim 33, wherein the plurality of layers includes at least two layers formed of active memory materials.

36. A vascular filter as set forth in claim 33, wherein at least one of the layers is a wire formed of a shape memory material and at least one of the layers is a braid formed of a shape memory material.

37. A vascular filter as set forth in claim 33, wherein the plurality of layers includes at least two layers braided together or one layer surrounded by a braid.

38. A vascular filter having first and second ends and a longitudinal central axis, the filter comprising:
a plurality of wire forms, each wire form having an initial end portion starting at the first end of the filter and extending in an axial direction substantially linearly therefrom, a central curved portion extending radially outwardly from the central axis and extending axially along the longitudinal central axis from the initial end portion to a maximum diameter section and then extending radially inwardly toward the central axis and extending axially the longitudinal central axis from the maximum diameter section, and a terminating end portion terminating at the second end of the filter and extending in an axial direction substantially linearly thereto.

39. The vascular filter of claim 38 wherein the initial end portions of each of the plurality of wire forms are affixed together and the terminating end portions of each of the plurality of wire forms are affixed together.

40. The vascular filter of claim 39 wherein the initial end portions and the terminating end portions are affixed together by crimping.

41. The vascular filter of claim 39 wherein each of the wire forms is made of a shape memory alloy.

42. The vascular filter of claim 41 wherein each of the wire forms includes a polymeric coating.

43. The vascular filter of claim 42 wherein the polymeric coating includes a therapeutic agent.

44. The vascular filter of claim 39 wherein the maximum diameter section of each of the plurality of wire forms is located a different axial distance from the first end of the vascular filter.

45. The vascular filter of claim 44 wherein the maximum diameter section of each of the plurality of wire forms is located about the same radial distance from the longitudinal central axis.

46. The vascular filter of claim 45 wherein each of the wire forms includes a polymeric coating.

47. The vascular filter of claim 46 wherein the polymeric coating includes a therapeutic agent.

48. The vascular filter of claim 45 wherein there are at least four wire forms.

49. The vascular filter of claim 48 wherein moving the first end of the vascular filter relative to the second end of the vascular filter to increase the axial distance between the first and second ends moves the maximum diameter sections of each of the plurality of wire forms radially towards the longitudinal central axis.
